(19) United States
(12) Patent Application Publication
(10) Pub. No.: US 2009/0093005 A1
(43) Pub. Date: Apr. 9, 2009

(54) PROTEIN-BASED BIOMARKERS FOR ABDOMINAL AORTIC ANEURYSM

(75) Inventors: David M. Smalley, Charlottesville, VA (US); Nancy L. Harthun, Charlottesville, VA (US); Klaus F. Ley, La Jolla, CA (US); Ian J. Sarembock, Cinncinnati, OH (US); Kristina Little, San Diego, CA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/245,322

(22) Filed: Oct. 3, 2008

Related U.S. Application Data
(60) Provisional application No. 60/997,865, filed on Oct. 5, 2007.

(51) Int. Cl. G01N 33/53; G01N 33/00; C12Q 1/00; C12Q 1/32; C12Q 1/48; C12Q 1/28; G01N 33/566

(52) U.S. Cl. 435/7.92; 436/86; 435/4; 435/26; 435/15; 435/28; 436/501

(57) ABSTRACT

The present invention encompasses compositions and methods useful for diagnosing subjects with abdominal aortic aneurysms. The invention relates to the use of protein biomarkers whose levels are different in subjects with abdominal aortic aneurysms relative to normal subjects.

### CROSS REFERENCE TO RELATED APPLICATIONS

This application is entitled to priority pursuant to 35 U.S. C §19(e) to U.S. provisional patent application No. 60/997,865, filed on Oct. 5, 2007. The entire disclosure of the afore-mentioned patent application is incorporated herein by reference.

### FIELD OF INVENTION

This invention relates generally to the field of diagnosing and monitoring abdominal aortic aneurysms using protein biomarkers.

### BACKGROUND

Death from ruptured abdominal aortic aneurysm (AAA) is the fifteenth leading killer of men and the twentieth leading killer of women in the United States. In 2000, 15,000 Americans died from abdominal aortic aneurysm (AAA). The disease has increased more than seven-fold since the 1950's. Increased age is one of the risk factors that can explain some of this trend. If detected and repaired by elective surgery, the survival is 93 to 95%. If repair is attempted after an AAA has ruptured, survival falls below 50%. There is a significant need to develop a laboratory test that is both inexpensive and readily available to screen patients for the presence of AAA. Safe treatment is available if the AAA is discovered prior to rupture, however, screening for AAA is in its infancy. The only screening tools currently employed are various types of cross-sectional imaging. This is costly and will probably never be cost-effective for large populations.

Prevalence of AAA varies with patient age and sex. An autopsy study documents overall prevalence of four percent. In patients between the ages of 60-64, the ratio of men to women with AAA is 11:1. This ratio drops to 3:1 between the ages of 85-89. Incidence of AAA in men peaks at age 80 (5.9%), and incidence of AAA in women peaks at age 90 (4.5%). Despite the fact that overall AAA prevalence in women is lower, they have significantly worse outcomes after rupture. 77% of ruptured AAA occur in men, (23% in women), however, women fare worse with a 65% mortality rate (42% in men). Due to this increased mortality, screening may be equally important in women. Currently, Medicare reimburses for an exploratory ultrasound scan to detect AAA only in men and women with a family history of AAA, and men with a smoking history of at least 100 cigarettes, at the age of 65 years, to be performed within 30 days of induction into Medicare.

The risk factors with the highest association with AAA are cigarette smoking, advanced age, family history, hypertension, and male sex. Cigarette smoking has the strongest association with AAAs that measure at least 4 cm in diameter (5.6 fold higher relative risk of AAA>4 cm in a recent study). This association implies that smoking accounted for 78% of the AAAs measuring this size in that study. This association between cigarette smoking and AAA>4 cm was also affected positively by the greater number of years spent smoking, indicating that this effect may be dose-dependent.

The biology of AAA is not well understood. Although there is a genetic component, and functional polymorphisms in the MMP9 gene are associated with AAA incidence, there is no consensus which genes should even be considered (Sandford et al., 2007, Eur. J. Vasc. Endovasc. Surg. 33:381-390; Ye, 2006, Cardiovasc. Res. 69:636-645). It is clear that patients suffering from Marfan's syndrome develop dissecting aneurysms of the aorta (ref), but not AAA. Genome-wide association studies are not available.

Because AAA is associated with a weakening of the aortic wall, gene products like matrix metalloproteinases (MMPs) have been considered as possible causative agents. This is supported by animal models. One commonly held view is that these enzymes together with the infiltration of inflammatory cells weaken the aortic wall until it fails structurally (Pearce and Shively, 2006, Ann. N. Y. Acad. Sci. 1085:117-32.:117-132). However, the evidence for MMPs is purely circumstantial.

There is a long felt need in the art for methods useful for diagnosing and monitoring the progression of AAA. The present invention satisfies these needs.

### SUMMARY OF THE INVENTION

The present invention is based on the discovery that there are detectable changes in the levels of various proteins in the blood plasma, more specifically in the blood plasma microparticles, of subjects with abdominal aortic aneurysms compared with subjects without abdominal aortic aneurysms.

The invention described herein was designed to study AAA patients by analyzing the proteome of the microparticles found in their blood plasma. By comparing the AAA plasma microparticle proteome with the microparticle proteome of healthy controls (Smalley et al., 2007, Thromb. Haemost. 97:67-80; Smalley and Ley, 2008, Clinical Laboratory 54:67-79) we hoped to gain insights into the pathophysiology of this important disease.

In healthy individuals, more than 90% of blood plasma microparticles are platelet-derived (Garcia et al., 2005, J. Proteome. Res. 4:1516-1521). Indeed, when comparing plasma-derived with platelet-derived microparticles, only 21 of 500 proteins were found to be differentially expressed (Smalley et al., 2007, Thromb. Haemost. 97:67-80). Based on these data, it was reasoned herein that in AAA patients, the number of microparticles might be augmented, because more microparticles might be shed from the diseased vessel wall than from the healthy vessel. In addition, we reasoned that their composition might be altered, because contributions from other cells might appear.

Shotgun sequencing proteomics is a powerful method that can find hundreds of proteins in a complex mixture (Garcia et al., 2005, J. Proteome. Res. 4:1516-1521; Coon et al., 2005, Biotechniques 38:519, 521, 523). Applying this technique to plasma has been difficult, because plasma contains many abundant, non-informative proteins like albumin (Anderson and Anderson, 2002, Mol. Cell Proteomics. 1:845-867). Because mass spectrometers can find proteins only when they are at least 1/1000 as common as the most abundant proteins, the signal from the top proteins drowns out possible signals from rare proteins. This is known as the dynamic range problem (Anderson and Anderson, 2002, Mol. Cell Proteomics. 1:845-867). Most plasma proteomics laboratories employ a depletion scheme to remove the six most abundant proteins (albumin, IgG, antitrypsin, IgA, transferrin, and haptoglobin) (Bjorhall et al., 2005, Proteomics. 5:307-317). However, this strategy reduces the dynamic range by only by one order of magnitude, but the difference between the most and least abundant proteins in human plasma is probably 10¹² to 10¹³ (Anderson and Anderson, 2002, Mol. Cell Proteomics. 1:845-867).

An alternative approach is described herein in which microparticles are physically isolated from human plasma (Smalley et al., 2007, Thromb. Haemost. 97:67-80). By its very nature, this process removes the most abundant plasma proteins. Using a high-resolution LTQ-FT instrument directly coupled to HPLC, we successfully found more than 500 proteins in plasma microparticles of AAA patients and risk factor-matched controls.

There is no biomarker for AAA available. MMP-9 has been isolated from AAA tissues in several studies. MMP-9 serum levels drop dramatically after AAA repair and high levels of MMP-9 are associated with AAA. However, elevated MMP9 levels are present in only half of the patients with AAA. The MMP-9 level does increase with increasing size of AAA. Furthermore, elevated MMP-9 is associated with many other pathologies. Although average MMP-9 levels are higher in AAA than in controls, MMP-9 is a very poor biomarker when it comes to predicting individual risk. Ninety-five of the 106 proteins identified have never been associated with AAA and therefore represent novel biomarkers that remain to be validated in larger cohorts of patients. Here, we identify 119 proteins that serve as potential biomarkers for those with previously undiagnosed aneurysms. One or more of these proteins may form the basis of a clinical diagnostic test to screen for AAA. We also found 29 proteins that are less abundant in MP from AAA patients. This may represent missing inhibitors, and may also reflect the “dilution” of normal MPs by pathological MPs. The biomarker discovery research strategy was based on the established identification of proteins contained in the patient's plasma microparticles, as described in U.S. patent application Ser. No. 11/935,048 and PCT US/2007/083722, filed Nov. 5 and 6, 2007, respectively.

The present invention provides AAA biomarkers. In one embodiment, the present invention encompasses the use of the proteins provided in the lists and Tables (Tables 1, 2, 3, 4, 5, and 6) of the Examples for diagnosing patients at risk for AAA. In one aspect, the groups of 148 proteins useful as biomarkers are summarized as follows:

1. Proteins found in AAA cases, but not in control subjects (2)

2. Proteins found in AAA cases, but in only a few non-AAA individuals controls (47)

3. Proteins found in most AAA cases and no controls (32)

4. Proteins found in most AAA cases and some controls (25)

5. Proteins potentially associated with hemolysis (13)

6. Proteins underexpressed in AAA (29)

The numbers described above are not limiting regarding the markers which can be used and are merely the ones identified to date using the methods of the invention.

The present invention further encompasses the use of combinations of any of the sets of biomarkers or combinations thereof, described herein for use in diagnosing, predicting the prognosis, predicting the onset or development, or monitoring the progression of AAA.

The present invention further encompasses the use of the sets of biomarkers disclosed herein for testing patients at risk for aneurysms.

The present invention encompasses methods of diagnosing an abdominal aortic aneurysm. The method comprises obtaining a biological sample from a test subject, measuring the level of at least one protein biomarker for abdominal aortic aneurysm disclosed herein, comparing the level of at least one protein biomarker associated with abdominal aortic aneurysm in the test subject with the level of the protein biomarker from an otherwise identical sample obtained from an unaffected subject or with a standard sample comprising a known amount of the protein biomarker. A higher or lower level of the protein biomarker in the sample from a test subject compared with the level of the protein biomarker in a sample from an unaffected subject or from a standard, is an indication that the test subject has an abdominal aortic aneurysm.

The present invention encompasses the use of various biological samples, including, but not limited to, tissue samples, biopsies, blood, plasma, saliva, feces, cerebrospinal fluid, semen, tears, and urine. In one aspect, the sample is plasma. In one aspect, the plasma is processed to obtain plasma-derived microparticles. In one aspect, the protein levels are measured using said microparticles.

In one embodiment, the present invention provides protein biomarkers which are found at higher levels in a subject with an abdominal aortic aneurysm compared with the level of in an unaffected subject. In one aspect, the protein biomarker found at higher levels in a subject with an abdominal aortic aneurysm include, but are not limited to, 14-3-3 protein epsilon, 14-3-3 protein eta, 14-3-3 protein gamma, 14-3-3 protein zeta/delta, 271 kDa protein, Actin-like protein 3, ACTN4 Alpha-actinin-4, Adenylyl cyclase-associated protein 1, Alpha-actinin-1, ARPC1B-Actin-related protein ⅔ complex subunit 1B, ARPC2 PNAS-139, ARPC4 Actin-related protein ⅔ complex subunit 4, Beta-parvin, Bridging integrator 2, Calpain-1 catalytic subunit, CANX Calnexin precursor, CAPZA2 F-actin capping protein subunit alpha-2, Carbonic anhydrase 2, CDNA FLJ45525 fis-clone BRTHA2026311-highly similar to Protein disulfide isomerase A6, Chloride intracellular channel protein 1, clathrin heavy chain 1, Coagulation factor XIII A chain precursor, Cofilin-1, Coronin-1C, COTL1 Coactosin-like protein, EDARADD ENO1P protein, EHD1 EH domain-containing protein 1, EHD3 EH domain-containing protein 3, Endoplasmin precursor, enolase 1, FHL1 Four and a half LIM domains 1 variant, FLNB Isoform 1 of Filamin-B, Glutathione S-transferase P, Glutathione transferase omega-1, Glyceraldehyde-3-phosphate dehydrogenase, GPX1 glutathione peroxidase 1 isoform 1, Heat shock protein 86 (Fragment), HSPA4 Heat shock 70 kDa protein 4, HSPC159 Galectin-related protein, Hypothetical protein, Hypothetical protein DKFZp761K0511, Hypothetical protein FLJ25678, IDH2 Isocitrate dehydrogenase [NADP]-mitochondrial precursor, Integrin-linked protein kinase 1, Isoform 1 of Alpha-parvin, Isoform 1 of Gelsolin precursor, Isoform 1 of Heat shock cognate 71 kDa protein, Isoform 1 of Vinculin, Isoform 2 of Unc-112-related protein 2, Isoform Beta-3B of Integrin beta-3 precursor, Isoform M1 of Pyruvate kinase isozymes M1/M2, Isoform SERCA3B of Sarcoplasmic/endoplasmic reticulum calcium ATPase 3, KCNK15 Potassium channel subfamily K member 15, Lactate dehydrogenase A, Leukocyte elastase inhibitor, L-lactate dehydrogenase B chain, LOC390006 similar to peptidylprolyl isomerase A isoform 1, LTBP1 Latent-transforming growth factor beta-binding protein-isoform IL precursor, LYAR Cell growth-regulating nucleolar protein, MAPRE2 Isoform 1 of Microtubule-associated protein RP/EB family member 2, MRPS30 28S ribosomal protein S31-mitochondrial precursor, Multimerin-1 precursor, Myosin regulatory light chain, Myosin regulatory light polypeptide 9 isoform b, Myosin-9, NME1 Nucleoside diphosphate kinase A, Peptidyl-prolyl cis-trans isomerase, Peptidylprolyl isomerase B precursor, Phosphoglycerate kinase 1, PINCH protein, Platelet glycoprotein V precursor, Pleckstrin, Protein disulfide-isomerase precursor, Protein DJ-1, PTGS1 Cyclooxygenase 1b3, Rab GDP dissociation inhibitor alpha, RAB6B Ras-related protein Rab-6B, RAC2 Ras-related C3 botulinum toxin substrate 2 precursor, Ras suppressor protein 1, Ras-related protein Rab-11B, Ras-related protein Rab-27B, RcTPI1 (Fragment), Rho GDP-dissociation inhibitor 2, RTN4 Isoform 1 of Reticulon-4, S100A4 Protein S100-A4, SELP P-selectin precursor, SEPT11 Septin-11, Serum deprivation-response protein, SLC2A3 Solute carrier family 2-facilitated glucose transporter member 3, SOD1 16 kDa protein, SPARC SPARC precursor, SPTBN5 Spectrin beta chain-brain 4, STXBP2 Syntaxin-binding protein 2, Superoxide dismutase [Mn]-mitochondrial precursor, TBXAS1 thromboxane A synthase 1 (platelet, cytochrome P450, family 5, subfamily A) isoform TXS-II, Thrombospondin-1 precursor, Thrombospondin-2 precursor, TPI1 Isoform 2 of Triosephosphate isomerase, TPM1 tropomyosin 1 alpha chain isoform 7, Transgelin-2, Transitional endoplasmic reticulum ATPase, Tropomyosin 4, Tubulin alpha-1 chain, Tubulin beta-1 chain, Tubulin beta-2C chain, and VASP Vasodilator-stimulated phosphoprotein. In one aspect, at least one of the protein biomarkers found at higher levels in a subject with an abdominal aortic aneurysm is FHL1 Four and a half LIM domains 1 variant. In another aspect, at least one of the protein biomarkers found at higher levels in a subject with an abdominal aortic aneurysm is COTL1 Coactosin-like protein.

In one aspect, the protein biomarkers which are found at higher levels in a subject with an abdominal aortic aneurysm include, but are not limited to proteins from the following groups or types of proteins-heat shock proteins, proteins related to extracellular matrix, proteins related to inflammation, proteins involved in metabolic processes, cytoskeletal proteins, proteins associated with endoplasmic reticulum, ion pumps and channels, proteins involved in angiogenesis, small GTPases, mast cell-associated proteins, platelet-associated proteins, surface receptor or associated proteins, proteins involved in calcium handling, oxidative stress proteins, mitochondrial proteins, signaling proteins, nuclear proteins, transcription factors, and proteins potentially associated with hemolysis.

In one aspect, the proteins potentially associated with hemolysis include, but are not limited to, ANK1 Isoform Er1 of Ankyrin-1, SPTA1 Spectrin alpha chain-erythrocyte, EPB41 Isoform 1 of Protein 4.1, Actin-like protein 2, Isoform 1 of Filamin-C, Filamin A alpha, F-actin capping protein alpha-1 subunit, PDZ and LIM domain protein 1, Fructose-bisphosphate aldolase A, Band 3 anion transport protein, EPB42 Isoform Long of Erythrocyte membrane protein band 4.2, Isoform 1 of F-actin capping protein subunit beta, and PKLR Isoform R-type of Pyruvate kinase isozymes R/L.

In one aspect, at least one of the protein biomarkers is one which is found at lower levels in a subject with an abdominal aortic aneurysm compared with the level of the protein biomarker in an unaffected subject. In one aspect, the protein biomarkers which are found at lower levels include, but are not limited to, 19 kDa protein, ALB protein (22434), ALB Protein (216773), Apolipoprotein A-I precursor, Apolipoprotein A-IV precursor, Apolipoprotein E precursor, Apolipoprotein F precursor, C4b-binding protein alpha chain precursor, Carbonic anhydrase 1, Clusterin precursor, Complement C4-A precursor, Factor VII active site mutant immunoconjugate, FLJ00385 protein (Fragment), Galectin-3-binding protein precursor, Hypothetical protein DKFZp686I04196 (Fragment), Ig kappa chain V-III region HAH precursor, Ig mu heavy chain disease protein, IGHA1 protein, IGHM protein, IGKV1-5 protein, IGLC1 protein, Isoform 2 of Reelin precursor, PREDICTED: HEG homolog 1, PRO2275, Syntenin-1, Transferrin receptor protein 1, Vitronectin precursor, and von Willebrand factor precursor.

In one embodiment of the invention, at least two protein biomarkers are compared. In one aspect, at least one of the protein biomarkers is one which is found at higher levels in a subject with an abdominal aortic aneurysm compared with the level of the protein biomarker in an unaffected subject. In one aspect, the protein biomarkers found at higher levels in a subject with an abdominal aortic aneurysm include, but are not limited to, 14-3-3 protein epsilon, 14-3-3 protein eta, 14-3-3 protein gamma, 14-3-3 protein zeta/delta, 271 kDa protein, Actin-like protein 3, ACTN4 Alpha-actinin-4, Adenylyl cyclase-associated protein 1, Alpha-actinin-1, ARPC1B-Actin-related protein ⅔ complex subunit 1B, ARPC2 PNAS-139, ARPC4 Actin-related protein ⅔ complex subunit 4, Beta-parvin, Bridging integrator 2, Calpain-1 catalytic subunit, CANX Calnexin precursor, CAPZA2 F-actin capping protein subunit alpha-2, Carbonic anhydrase 2, CDNA FLJ45525 fis-clone BRTHA2026311-highly similar to Protein disulfide isomerase A6, Chloride intracellular channel protein 1, clathrin heavy chain 1, Coagulation factor XIII A chain precursor, Cofilin-1, Coronin-1C, COTL1 Coactosin-like protein, EDARADD ENO1P protein, EHD1 EH domain-containing protein 1, EHD3 EH domain-containing protein 3, Endoplasmin precursor, enolase 1, FHL1 Four and a half LIM domains 1 variant, FLNB Isoform 1 of Filamin-B, Glutathione S-transferase P, Glutathione transferase omega-1, Glyceraldehyde-3-phosphate dehydrogenase, GPX1 glutathione peroxidase 1 isoform 1, Heat shock protein 86 (Fragment), HSPA4 Heat shock 70 kDa protein 4, HSPC159 Galectin-related protein, Hypothetical protein, Hypothetical protein DKFZp761K0511, Hypothetical protein FLJ25678, IDH2 Isocitrate dehydrogenase [NADP]-mitochondrial precursor, Integrin-linked protein kinase 1, Isoform 1 of Alpha-parvin, Isoform 1 of Gelsolin precursor, Isoform 1 of Heat shock cognate 71 kDa protein, Isoform 1 of Vinculin, Isoform 2 of Unc-112-related protein 2, Isoform Beta-3B of Integrin beta-3 precursor, Isoform M1 of Pyruvate kinase isozymes M1/M2, Isoform SERCA3B of Sarcoplasmic/endoplasmic reticulum calcium ATPase 3, KCNK15 Potassium channel subfamily K member 15, Lactate dehydrogenase A, Leukocyte elastase inhibitor, L-lactate dehydrogenase B chain, LOC390006 similar to peptidylprolyl isomerase A isoform 1, LTBP1 Latent-transforming growth factor beta-binding protein-isoform 1L precursor, LYAR Cell growth-regulating nucleolar protein, MAPRE2 Isoform 1 of Microtubule-associated protein RP/EB family member 2, MRPS30 28S ribosomal protein S31-mitochondrial precursor, Multimerin-1 precursor, Myosin regulatory light chain, Myosin regulatory light polypeptide 9 isoform b, Myosin-9, NME1 Nucleoside diphosphate kinase A, Peptidyl-prolyl cis-trans isomerase, Peptidylprolyl isomerase B precursor, Phosphoglycerate kinase 1, PINCH protein, Platelet glycoprotein V precursor, Pleckstrin, Protein disulfide-isomerase precursor, Protein DJ-1, PTGS1 Cyclooxygenase 1b3, Rab GDP dissociation inhibitor alpha, RAB6B Ras-related protein Rab-6B, RAC2 Ras-related C3 botulinum toxin substrate 2 precursor, Ras suppressor protein 1, Ras-related protein Rab-11B, Ras-related protein Rab-27B, RcTPI1 (Fragment), Rho GDP-dissociation inhibitor 2, RTN4 Isoform 1 of Reticulon-4, S100A4 Protein S100-A4, SELP P-selectin precursor, SEPT11 Septin-11, Serum deprivation-response protein, SLC2A3 Solute carrier family 2-facilitated glucose transporter member 3, SOD1 16 kDa protein, SPARC SPARC precursor, SPTBN5 Spectrin beta chain-brain 4, STXBP2 Syntaxin-binding protein 2, Superoxide dismutase [Mn]-mitochondrial precursor, TBXAS1 thromboxane A synthase 1 (platelet, cytochrome P450, family 5, subfamily A) isoform TXS-II, Thrombospondin-1 precursor, Thrombospondin-2 precursor, TPI1 Isoform 2 of Triosephosphate isomerase, TPM1 tropomyosin 1 alpha chain isoform 7, Transgelin-2, Transitional endoplasmic reticulum ATPase, Tropomyosin 4, Tubulin alpha-1 chain, Tubulin beta-1 chain, Tubulin beta-2C chain, and VASP Vasodilator-stimulated phosphoprotein. In one aspect, at least one of the protein biomarkers found at higher levels in a subject with an abdominal aortic aneurysm is FHL1 Four and a half LIM domains 1 variant. In another aspect, at least one of the protein biomarkers found at higher levels in a subject with an abdominal aortic aneurysm is COTL1 Coactosin-like protein.

In one aspect when at least two protein biomarkers are compared, at least one protein biomarker which is found at higher levels in a subject with an abdominal aortic aneurysm includes, but is not limited to, heat shock proteins, proteins related to extracellular matrix, proteins related to inflammation, proteins involved in metabolic processes, cytoskeletal proteins, proteins associated with endoplasmic reticulum, ion pumps and channels, proteins involved in angiogenesis, small GTPases, mast cell-associated proteins, platelet-associated proteins, surface receptor or associated proteins, proteins involved in calcium handling, oxidative stress proteins, mitochondrial proteins, signaling proteins, nuclear proteins, transcription factors, and proteins potentially associated with hemolysis.

In one aspect, the proteins potentially associated with hemolysis include, but are not limited to, ANK1 Isoform Er1 of Ankyrin-1, SPTA1 Spectrin alpha chain-erythrocyte, EPB41 Isoform 1 of Protein 4.1, Actin-like protein 2, Isoform 1 of Filamin-C, Filamin A alpha, F-actin capping protein alpha-1 subunit, PDZ and LIM domain protein 1, Fructose-bisphosphate aldolase A, Band 3 anion transport protein, EPB42 Isoform Long of Erythrocyte membrane protein band 4.2, Isoform 1 of F-actin capping protein subunit beta, and PKLR Isoform R-type of Pyruvate kinase isozymes R/L.

In one embodiment of the invention where at least two protein biomarkers are compared, at least one of the protein biomarkers is one which is found at lower levels in a subject with an abdominal aortic aneurysm compared with the level of the protein biomarker in an unaffected subject. In one aspect, the protein biomarkers which are found at lower levels include, but are not limited to, 19 kDa protein, ALB protein (22434), ALB Protein (216773), Apolipoprotein A-I precursor, Apolipoprotein A-IV precursor, Apolipoprotein E precursor, Apolipoprotein F precursor, C4b-binding protein alpha chain precursor, Carbonic anhydrase 1, Clusterin precursor, Complement C4-A precursor, Factor VII active site mutant immunoconjugate, FLJ00385 protein (Fragment), Galectin-3-binding protein precursor, Hypothetical protein DKFZp686I04196 (Fragment), Ig kappa chain V-III region HAH precursor, Ig mu heavy chain disease protein, IGHA1 protein, IGHM protein, IGKV1-5 protein, IGLC1 protein, Isoform 2 of Reelin precursor, PREDICTED: HEG homolog 1, PRO2275, Syntenin-1, Transferrin receptor protein 1, Vitronectin precursor, and von Willebrand factor precursor.

In one embodiment, at least one of the biomarkers is one which is found at higher levels in a subject with an abdominal aortic aneurysm and at least one of the protein biomarkers is one which is found at lower levels in a subject with an abdominal aortic aneurysm compared with the level of the protein biomarker in an unaffected subject.

In one embodiment, the subject is a human. In one aspect, the test subject is at risk for developing an abdominal aortic aneurysm. In one aspect, the test subject has at least one risk factor for developing an abdominal aortic aneurysm. In one aspect, the risk factors include, but are not limited to, high blood pressure, smoking, high cholesterol, emphysema, genetic factors, age, and male gender. One of ordinary skill in the art will understand which risk factors can be used to select a subject for diagnosis. In one aspect, the test subject is asymptomatic for an abdominal aortic aneurysm. In another aspect, the test subject is symptomatic for an abdominal aortic aneurysm. One of ordinary skill in the art can use such risk factors to help determine when to first test a subject at risk and how often to re-test such a subject if the subject is negative for any changes in biomarkers associated with abdominal aortic aneurysm.

One of ordinary skill in the art will appreciate that depending on the particular protein or group of protein biomarkers being measured, different assays can be used for measurement of the biomarker. In one aspect, the protein biomarker levels are measured using techniques including, but not limited to, flow cytometry, western blots, immunoblots, ELISA, MS/MS spectroscopy, and biological activity assays. If more than one protein is being measured, more than one type of assay can be used.

The present invention further provides methods of monitoring the progression of an abdominal aortic aneurysm in a subject previously diagnosed with an abdominal aortic aneurysm. By progression is meant a change in the aneurysm such as an increase in size. The method includes obtaining samples at various times from the subject and comparing the protein levels to an early measurement in that subject or to a standard. The invention provides methods for measuring the level of at least one protein biomarker associated with an abdominal aortic aneurysm in a first biological sample obtained from the test subject to determine an initial level of at least one protein biomarker and then measuring the level of the protein biomarker(s) in a second otherwise identical biological sample obtained from the subject at a later point in time. Then, the level of the protein biomarker in the first biological sample is compared with the level of the same protein biomarker in the second otherwise identical biological sample obtained from said subject. Any change in the level of the protein biomarker in the second otherwise identical biological sample is used to determine whether there is a change in the abdominal aortic aneurysm. The invention encompasses making multiple measurements at various times following the initial diagnosis or once a baseline is set. One of ordinary skill in the art will be able to determine how often to take samples from the subject and which biomarkers are to be measured. Such determinations will be based on criteria such as the age, gender, smoking habits, and physical state of the subject. Other criteria for being at risk include hypertension, and genetic factors. In one aspect at least one of protein biomarkers is measured which is found at higher levels in a subject with an abdominal aortic aneurysm compared with the level of the protein biomarker in an unaffected subject. In another aspect, at least one of the protein biomarkers is one which is found at lower levels in a subject with an abdominal aortic aneurysm compared with the level of said protein biomarker in an unaffected subject. In one aspect, at least two protein biomarkers are measured.

The present invention further encompasses monitoring a subject who has been treated for an abdominal aortic aneurysm.

The present invention further encompasses a kit for measuring protein biomarkers of the invention. In one aspect, the kit provides standardized samples. In one aspect, the kit provides reagents for measuring the protein biomarkers. In one aspect, the kit provides an instructional material.

Various aspects and embodiments of the invention are described in further detail below.

### DETAILED DESCRIPTION

### Abbreviations and Acronyms

AAA means abdominal aortic aneurysm
AIM means Apoptosis Inhibitor in Macrophages
C4BP means Complement Component C4 Binding Protein
FCGBP means Fc fragment of IgG binding protein
ICAT means Isotope-Coded Affinity Tag
LC/MS means liquid chromatography/mass spectrometry
MPs means Microparticles
PAGE means Polyacrylamide gel electrophoresis
PBS means Phosphate buffered saline
PPP means Platelet-Poor Plasma
PRP means Platelet-Rich Plasma
SDS means sodium dodecyl sulfate
vWF means von Willebrand Factor

### Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

As used herein, the term “affected cell” refers to a cell of a subject afflicted with a disease or disorder, which affected cell has an altered phenotype relative to a subject not afflicted with a disease or disorder. Cells or tissue are “affected” by a disease or disorder if the cells or tissue have an altered phenotype relative to the same cells or tissue in a subject not afflicted with a disease or disorder.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

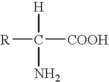

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

The term “basic” or “positively charged” amino acid as used herein, refers to amino acids in which the R groups have a net positive charge at pH 7.0, and include, but are not limited to, the standard amino acids lysine, arginine, and histidine.

As used herein, the term “biologically active fragments” or “bioactive fragment” of the polypeptides encompasses natural or synthetic portions of the full-length protein that are capable of specific binding to their natural ligand or of performing the function of the protein.

A “biomarker” is a specific biochemical in the body which has a particular molecular feature that makes it useful for measuring the progress of disease or the effects of treatment, or for measuring a process of interest.

A “compound,” as used herein, refers to a polypeptide, an isolated nucleic acid, or other agent used, identified, or isolated in the method of the invention.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting AAA or a risk or propensity for development of AAA. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “microparticle”, as used herein, refers to any protein containing particle less than 1 micron in diameter with a molecular weight of over 100,000 daltons. These include various lipoproteins and membrane vesicles released from cells.

As used herein, a “peptide” encompasses a sequence of 2 or more amino acid residues wherein the amino acids are naturally occurring or synthetic (non-naturally occurring) amino acids covalently linked by peptide bonds. No limitation is placed on the number of amino acid residues which can comprise a protein's or peptide's sequence. As used herein, the terms “peptide,” polypeptide,” and “protein” are used interchangeably. Peptide mimetics include peptides having one or more of the following modifications:

1. peptides wherein one or more of the peptidyl —C(O)NR— linkages (bonds) have been replaced by a non-peptidyl linkage such as a —CH₂₋carbamate linkage (—CH₂OC(O)NR—), a phosphonate linkage, a —CH₂₋sulfonamide (—CH₂₋S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

“Plurality” means at least two.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups. As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “purified” relates to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

The term “standard,” as used herein, refers to something used for comparison. For example, a standard can be a known standard agent or compound which is administered or added to a control sample and used for comparing results when measuring said compound in a test sample. In one aspect, the standard compound is added or prepared at an amount or concentration that is equivalent to a normal value for that compound in a normal subject. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

### Embodiments of the Invention

The present invention provides methods and compositions useful for diagnosing AAA, monitoring the progression or changes in AAA, and determining the prognosis for subjects with AAA. It also encompasses assessing the risk for developing AAA. To that end, the present invention provides a series of protein biomarkers useful for diagnosing AAA, monitoring the progression or changes in AAA, and determining the prognosis for subjects with AAA. In one aspect, a biomarker is found at higher levels. In one aspect, a biomarker is found at lower levels. In one aspect, a combination of biomarkers can be used, some of which are found at higher levels in subjects with AAA and some of which are at lower levels in subjects with AAA. One of ordinary skill in the art can determine which sets of markers to use and the parameters for determining whether a marker is found at a higher level or at a lower level, relative to a control subject or to a standard. In one aspect, the higher or lower level of a protein biomarker, compared to the level in a control subject, can be at least about 1%, 2%, 5%, 7.5%, 10%, 15%, 20%, or at least about 25%.

Risk factors for developing an aortic aneurysm include, but are not limited to, high blood pressure, smoking, high cholesterol, emphysema, genetic factors, age, and male gender. In one aspect, a subject with one or more risk factors can be tested for an AAA using the methods of the invention. One of ordinary skill in the art will understand which risk factors should be considered when recommend such a test. The present invention is also useful for monitoring the progression or change in size of an AAA in a subject who has been diagnosed with AAA. The present invention can also be used to monitor a subject where surgical intervention has been used to correct a AAA.

Most abdominal aortic aneurysms produce no symptoms (they are asymptomatic). They are often incidentally discovered when abdominal ultrasounds and/or CT scan studies are ordered for other conditions. When they produce symptoms, the most common symptom is pain. The pain typically has a deep quality as if it is boring into the person. It is felt most prominently in the middle of the abdomen and can radiate to the back. The pain is usually steady but may be relieved by changing position. The person may also become aware of an abnormally prominent abdominal pulsation.

Abdominal aortic aneurysms can remain asymptomatic or produce mild to moderate symptoms for years. However, a rapidly expanding abdominal aneurysm can cause sudden onset of severe, steady, and worsening middle abdominal and back pain. A rapidly expanding aneurysm is also at imminent risk of rupture.

The methods of the present invention can be used in subjects who are asymptomatic, and as well those who are symptomatic for AAA or for a ruptured AAA. Symptoms for rupture of an AAA include, but are not limited to, pulsating sensation in the abdomen, pain in the abdomen or back (severe, sudden, persistent, or constant, which may radiate to groin, buttocks, or legs), abdominal rigidity, anxiety, nausea and vomiting, clammy skin, rapid heart rate when rising to a standing position, shock, and abdominal mass.

In one embodiment, the present invention provides multiple protein biomarkers useful for diagnosing AAA, predicting the onset or development of AAA, and monitoring the progression of AAA once diagnosed.

It will be understood by the skilled artisan that markers may be used singly or in combination with other markers (as well as the use of ratios of markers) for any of the uses, e.g., diagnosing AAA or monitoring the progression of AAA, disclosed herein. For example, once a subject is diagnosed with AAA using the methods of the invention and the levels of the biomarkers indicated that a small aneurysm is present, one of ordinary skill in the art may want to continue monitoring the subject to determine if there is a progression, i.e., change in size, to help make a decision as to when or if the aneurysm should be treated. Such a progression can be associated with a greater change in the level of one or more biomarkers compared with a previous measurement, or with a change in the ratio of biomarkers. These measurements may also be used in conjunction with imaging techniques. Various combinations of markers are also useful for staging and or prognosis and for monitoring subjects after treatment.

In one embodiment, the present invention provides methods for measuring proteins using isolated microparticles from plasma which has been depleted of platelets. In one aspect, the method provides for identifying and analyzing biomarkers associated with AAA using microparticles. Upon activation, many different cell types release microparticles. It is likely that the composition and number of microparticles in the plasma may be important markers for disease predisposition, diagnosis, and progression. In one aspect, the biomarkers are proteins and peptides, or homologs or fragments thereof. In one aspect, the presence of a biomarker identified by the methods of the invention, or a difference in the level of the biomarker relative to a normal control level, is indicative of an abdominal aortic aneurysm. In one embodiment, the present invention provides diagnostic assays for abdominal aortic aneurysms using biomarkers identified by the methods of the invention. The invention also encompasses the identification of novel proteins whose levels change in subjects with abdominal aortic aneurysms.

Protein biomarkers of the present invention may be measured using any applicable technique for detecting and quantifying the amount or level of a protein in a sample. Such techniques include, but are not limited to, flow cytometry, western blots, immunoblots, ELISA, MS/MS spectroscopy, and biological activity assays. Various assays are used to detect, identify, and quantify proteins. Some assays use a light-producing reaction or radioactivity to generate a signal. Other assays produce an amplified colored signal with enzymes and chromogenic substrates. One of ordinary skill in the art will appreciate how each protein biomarker can be measured and which type of assay is available for measuring it.

Fragments of the protein biomarkers of the invention are encompassed by the methods of the invention. For example, MS/MS spectroscopy can identify the fragments and can be used. Additionally, in cases where it is known that, in addition to the full length biomarker, there is a certain fragment which routinely occurs, then antibodies can made which are directed against that fragment, and the antibody can be used in the various types of immunoassays that are available.

Antibody reagents can be used in assays to detect biomarkers of the invention in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser-induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

A signal from a direct or indirect label can be analyzed, for example, using a spectrophotometer to detect color from a chromogenic substrate; a radiation counter to detect radiation such as a gamma counter for detection of 125I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocyanate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibody probes to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

The invention also includes a kit comprising standard protein biomarkers or compositions comprising added known amounts of protein biomarkers of the invention, or biological samples with known amounts of the protein biomarkers, and an instructional material that describes using the composition or measuring the protein biomarker(s) of interest. In another embodiment, this kit comprises a (preferably sterile) solvent suitable for dissolving or suspending the composition of the invention as well as reagents for measuring the protein biomarker.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

### Materials and Methods

Isolation of platelets platelet-derived MPs, and plasma-derived MPs. Platelets and platelet-derived MPs were isolated as previously described. Briefly, human blood was collected by venipuncture into 1/10 volume of an acid-citrate-dextrose (85 mM trisodium citrate, 83 mM dextrose, and 21 mM citric acid) solution. Platelet-rich plasma (PRP) was obtained by centrifugation at 110×g for 15 minutes. Platelets were pelleted by centrifugation at 710×g for 15 minutes, and the supernatant, platelet-poor plasma (PPP), was retained for isolation of plasma MPs (see below).

Plasma-derived MPs were isolated by gel filtration chromatography followed by ultracentrifugation. Briefly, the platelet-poor plasma (PPP) generated above was centrifuged an additional two times to remove residual cells and cell debris at 710×g and 25° C. for 15 minutes. This plasma was then applied to a Sephacryl® S-500 HR (GE Healthcare, Piscataway, N.J.) gel filtration column and MP-containing fractions were concentrated by ultracentrifugation at 150,000×g for 90 minutes at 10° C.

Sample preparation for unlabeled protein analysis. MPs were resuspended in a minimal volume of PBS (phosphate buffered saline, pH 7.4) and a small aliquot was taken for protein analysis using the Micro BCA Protein Assay (Pierce Biotechnology, Inc., Rockford, Ill.). Forty microliters of plasma microparticles, resuspended with PBS to 40 μL, were mixed with 10 μL of 5× SDS-PAGE loading buffer (0.5 M Tris, pH 6.8, 10% SDS, 38% glycerol, 0.1% bromophenol blue). The separate samples (50 μL each) were heated to 95° C. for 5 minutes, allowed to cool to room temperature, and centrifuged for 2 minutes at 14,000 rpm prior to loading onto the gel. Microparticle proteins were electrophoresed approximately 1 cm into a 7.5% acrylamide SDS-PAGE using a Mini-gel system (BioRad, Hercules, Calif.) at 150 V. The acrylamide gel section containing the proteins was cut out and placed in fixative (50% methanol, 12% acetic acid, 0.05% formalin) for 2 hours. The in-gel tryptic digestion of the lanes and the peptide extraction was performed. The extracted peptide solutions were lyophilized and reconstituted to 20 μL with 0.1% acetic acid for mass spectrometry analysis. Three sets of platelet- and plasma-derived MP peptides were generated, and each of these samples was analyzed by LC/MS twice.

Liquid chromatography/mass spectrometry (LC/MS) and protein identification. Samples were loaded onto a 360 μm o.d.×75 μm i.d. microcapillary fused silica precolumn packed with irregular 5-20 μm C 18 resin. After sample loading, the precolumn was washed with 0.1% acetic acid for 15 minutes to remove any buffer salts or gel contaminants. The precolumn was then connected to a 360 μm o.d.×50 μm i.d. analytical column packed with regular 5 μm C18 resin constructed with an integrated electrospray emitter tip. Samples were gradient eluted with an 1100 series binary HPLC solvent delivery system (Agilent, Palo Alto, Calif.) directly into a Finnigan LTQ-FT ion trap mass spectrometer (Thermo Electron Corp, San Jose, Calif.) at a flow rate of 60 nL/min. The HPLC stepwise gradient used was initially 100% A, 5% B at 5 minutes, 50% B at 220 minutes, 100% B at 240 minutes, and restored to 100% A at 280 minutes (solvent A=0.1 M acetic acid, solvent B=70% acetonitrile in 0.1 M acetic acid). The LTQ-FT mass spectrometer was operated in the data-dependent mode in which first an initial MS scan recorded the mass to charge (m/z) ratios of ions over the mass range 300-2000 Da, and then the 10 most abundant ions were automatically selected for subsequent collisionally-activated dissociation and an MS/MS spectrum recorded.

All MS/MS data were searched against a human protein database downloaded from the NCBI website using the SEQUEST® program (Thermo Electron Corp.). For unlabeled peptides, a static modification of 57 Da for cysteine residues was employed in the search parameters. Peptide identifications were made using a first-pass filtering of standard criteria as previously described, including cross correlation values ≧2.0 (+1 charge), 2.2 (+2 charge) and 3.5 (+3 charge) and all peptides must be fully tryptic. Protein assignments required at least 2 MS/MS spectra matches that passed the above criteria. Manual validation of at least one MS/MS spectrum-peptide sequence match per protein was performed for all proteins that were determined to be differentially expressed.

Comparative analysis of unlabeled peptides using Spectral Count. All search results not passing the first-pass filter were eliminated. The number of spectra for each peptide was determined, and the number of total proteins detected were calculated. If any protein had a spectral count of less than 2 for either the plasma MPs, it was eliminated from that group. Only proteins with an overall spectral count of 10 or greater were analyzed further by this method. The ratio of spectra from the plasma MP versus the platelet MP was calculated, log 2 transformed, and then adjusted for an overall ratio score of 0.00 excluding vWF-containing peptides. The standard deviation (SD) of the log score was calculated, and all proteins that were over 3 SD above (or below) the mean were considered to be enriched with “high confidence”. Proteins with log 2 scores between 2 and 3 SD greater than the mean were classified as possible candidates that should be examined further and referred to as “low confidence”.

Enrichment Analysis

Gene set enrichment analysis (GSEA) (Subramanian, Tamayo, et al. (2005, PNAS 102, 15545-15550) and Mootha, Lindgren, et al. (2003, Nat Genet 34, 267-273)) was modified providing a rather unbiased approach to identify functional categories of proteins overrepresented in AAA patients. Genes coding for the detected proteins were identified and used for the analysis. Similarly, instead of gene expression levels the number of spectral counts was applied. Based on the Affymetrix HG 133 2 Plus gene list, enrichment was analyzed employing the curated gene sets provided online by the Broad Institute. Significance of enrichment was assessed based on false discovery rate and P value.

Other techniques useful in the present application are known in the art and may be found, for example, in International Patent Publication No. WO 2008/091948 (Smalley et al., published Jul. 31, 2008; International Application No. PCT/US2008/051801).

### Results

The proteome of these particles in patients with known AAA was compared with age- and sex-matched controls that were also matched for risk factors. Each plasma sample was processed to obtain microparticles and analyzed by MS/MS using a ThermoFinnigan LTQ-FT instrument. Over 32,000 peptides were detected using the Sequest algorithm with a cutoff of XCOR according to standard practices, dependent on the charge state. These peptides mapped to 761 different proteins (gene products).

T-test-based statistical comparison showed that 148 proteins were expressed differently between AAA cases (119 overexpressed) and controls (29 underexpressed) at a level of p<0.01. These p values are not adjusted for multiple comparisons.

The proteins were then grouped into five groups, in the order of decreasing certainty, with the number found indicated in parentheses:
1. Proteins found in all AAA cases and no controls (2)
2. Proteins found in all AAA cases and some controls (47)
3. Proteins found in most AAA cases and no controls (32)
4. Proteins found in most AAA cases and some controls (25)
5. Proteins potentially associated with hemolysis (13) Negative biomarkers (levels decreased in AAA) formed a sixth group:
6. Proteins underexpressed in AAA (29)

Hemolysis can be an artifact of blood drawing, but may also reflect a real biomarker that distinguishes AAA from controls. Therefore, these proteins are also claimed as potential biomarkers for AAA.

Among the 106 positive biomarker candidates detected that were not associated with hemolysis, two represent potential novel proteins, where database entries indicate just a genetic locus with no known protein. These are highlighted in green. Of the remaining 104 proteins, 11 had previously been associated with AAA. This discovery of known proteins validates the method. Because these 11 proteins have not previously been identified as biomarkers for AAA, they are encompassed within the methods of the invention.

Many of the differentially expressed microparticle proteins are associated with the following processes and cells that may actually be involved in AAA pathophysiology (see list below).

Blood samples were centrifuged to remove platelets, residual cells, and cell debris. Gel filtration chromatography was used to isolate plasma microparticles (MPs). Following a total protein assay, the MPs were prepared for unlabeled shotgun peptide analysis. The MPs were solubilized in a detergent solution and then loaded onto an acrylamide gel for brief electrophoresis followed by in-gel tryptic digestion. The resulting samples consisted of a mixture of MP protein tryptic peptides. All samples were prepared in duplicate aliquots to account for variability. The prepared tryptic peptide samples were analyzed by online liquid chromatography/tandem mass spectrometry (LC/MS-MS) using a Thermo LTQ-FT instrument. Peptides were fractionated by reverse phase LC, eluted into an electrospray ionization (ESI) source and the ions generated were analyzed by an automated program that yields MS (peptide mass and abundance) and MS-MS (selected peptide fragmentation) spectra. Comparative analyses of two or more MP samples will be accomplished by sequential individual analyses of unlabeled samples. The MS-MS spectra were searched with the SEQUEST algorithm against a human protein database to identify the peptides and their protein sources. Candidate differentially-expressed peptides/proteins were identified by multiple statistical tests based on spectral counts. The number of MS-MS spectra is related to peptide abundance. Only proteins that distinguish the AAA sample set from controls at a significance level p-value <0.01 or better are included in this invention disclosure.

Alphabetical list of proteins useful as positive biomarkers for AAA (overexpressed/higher levels in AAA)
14-3-3 protein epsilon
14-3-3 protein eta
14-3-3 protein gamma
14-3-3 protein zeta/delta
271 kDa protein
Actin-like protein 3
ACTN4 Alpha-actinin-4
Adenylyl cyclase-associated protein 1
Alpha-actinin-1
ARPC1B Actin-related protein ⅔ complex subunit 1B
ARPC2 PNAS-139
ARPC4 Actin-related protein ⅔ complex subunit 4
Beta-parvin
Bridging integrator 2
Calpain-1 catalytic subunit
CANX Calnexin precursor
CAPZA2 F-actin capping protein subunit alpha-2
Carbonic anhydrase 2
CDNA FLJ45525 fis-clone BRTHA2026311-highly similar to Protein disulfide isomerase A6
Chloride intracellular channel protein 1
clathrin heavy chain 1
Coagulation factor XIII A chain precursor
Cofilin-1
Coronin-1C
COTL1 Coactosin-like protein
EDARADD ENO1P protein
EHD1 EH domain-containing protein 1
EHD3 EH domain-containing protein 3
Endoplasmin precursor
enolase 1
FHL1 Four and a half LIM domains 1 variant
FLNB Isoform 1 of Filamin-B
Glutathione S-transferase P
Glutathione transferase omega-1
Glyceraldehyde-3-phosphate dehydrogenase
GPX1 glutathione peroxidase 1 isoform 1
Heat shock protein 86 (Fragment)
HSPA4 Heat shock 70 kDa protein 4
HSPC159 Galectin-related protein
Hypothetical protein
Hypothetical protein DKFZp761K0511
Hypothetical protein FLJ25678
IDH2 Isocitrate dehydrogenase [NADP]-mitochondrial precursor
Integrin-linked protein kinase 1
Isoform 1 of Alpha-parvin
Isoform 1 of Gelsolin precursor
Isoform 1 of Heat shock cognate 71 kDa protein
Isoform 1 of Vinculin
Isoform 2 of Unc-112-related protein 2
Isoform Beta-3B of Integrin beta-3 precursor
Isoform M1 of Pyruvate kinase isozymes M1/M2
Isoform SERCA3B of Sarcoplasmic/endoplasmic reticulum calcium ATPase 3
KCNK15 Potassium channel subfamily K member 15
lactate dehydrogenase A
Leukocyte elastase inhibitor
L-lactate dehydrogenase B chain
LOC390006 similar to peptidylprolyl isomerase A isoform 1
LTBP1 Latent-transforming growth factor beta-binding protein, isoform 1L precursor
LYAR Cell growth-regulating nucleolar protein
MAPRE2 Isoform 1 of Microtubule-associated protein RP/EB family member 2
MRPS31 28S ribosomal protein S31, mitochondrial precursor
Multimerin-1 precursor
Myosin regulatory light chain
Myosin regulatory light polypeptide 9 isoform b
Myosin-9
NME1 Nucleoside diphosphate kinase A
Peptidyl-prolyl cis-trans isomerase
Peptidylprolyl isomerase B precursor
Phosphoglycerate kinase 1
PINCH protein
Platelet glycoprotein V precursor
Pleckstrin
Protein disulfide-isomerase precursor
Protein DJ-1
PTGS1 Cyclooxygenase 1b3
Rab GDP dissociation inhibitor alpha
RAB6B Ras-related protein Rab-6B
RAC2 Ras-related C3 botulinum toxin substrate 2 precursor
Ras suppressor protein 1
Ras-related protein Rab-11B
Ras-related protein Rab-27B
RcTPI1 (Fragment)
Rho GDP-dissociation inhibitor 2
RTN4 Isoform 1 of Reticulon-4
S100A4 Protein S100-A4
SELP P-selectin precursor
SEPT11 Septin-11
Serum deprivation-response protein
SLC2A3 Solute carrier family 2, facilitated glucose transporter member 3
SOD1 16 kDa protein
SPARC SPARC precursor
SPTBN5 Spectrin beta chain, brain 4
STXBP2 Syntaxin-binding protein 2
Superoxide dismutase [Mn], mitochondrial precursor
TBXAS1 thromboxane A synthase 1 (platelet, cytochrome P450, family 5, subfamily A) isoform TXS-II
Thrombospondin-1 precursor
Thrombospondin-2 precursor
TPI1 Isoform 2 of Triosephosphate isomerase
TPM1 tropomyosin 1 alpha chain isoform 7
Transgelin-2
Transitional endoplasmic reticulum ATPase
Tropomyosin 4
Tubulin alpha-1 chain
Tubulin beta-1 chain
Tubulin beta-2C chain
VASP Vasodilator-stimulated phosphoprotein

### Alphabetical List of Proteins Useful as Negative Biomarkers for AAA (Underexpressed/Lower Levels in AAA; See Table 6)

19 kDa protein
ALB protein (accession number 22434; p val 0.00012 87; IP100022434)
ALB protein (accession number 216773, p val 6.21E-06; IP100216773)
Apolipoprotein A-I precursor
Apolipoprotein A-IV precursor
Apolipoprotein E precursor
Apolipoprotein F precursor
C4b-binding protein alpha chain precursor
Carbonic anhydrase 1
Clusterin precursor
Complement C4-A precursor
Factor VII active site mutant immunoconjugate
FLJ00385 protein (Fragment)
Galectin-3-binding protein precursor
Hypothetical protein DKFZp686I04196 (Fragment)
Ig kappa chain V-III region HAH precursor
Ig mu heavy chain disease protein
IGHA1 protein
IGHM protein
IGKV1-5 protein
IGLC1 protein
Isoform 2 of Reelin precursor
PREDICTED: HEG homolog 1
PRO2275
Syntenin-1
Transferrin receptor protein 1
Vitronectin precursor
von Willebrand factor precursor

The following proteins identified to be more abundant in microparticles from AAA patients can be grouped as follows:

### Heat Shock Proteins:

Hypothetical protein DKFZp761 K0511
Heat shock protein 86 (Fragment)
Hypothetical protein
HSPA4 Heat shock 70 kDa protein 4
Endoplasmin precursor
Isoform 1 of Heat shock cognate 71 kDa protein

### Related to Extracellular Matrix:

Hypothetical protein DKFZp761 K0511
Heat shock protein 86 (Fragment)
Protein disulfide-isomerase precursor
Thrombospondin-1 precursor
Coagulation factor XIII A chain precursor

### Related to Inflammation:

Hypothetical protein DKFZp761 K0511
Heat shock protein 86 (Fragment)
Coagulation factor XIII A chain precursor
Clathrin heavy chain 1
Bridging integrator 2
FHL1 Four and a half LIM domains 1 variant
HSPC159 Galectin-related protein
TBXAS1 thromboxane A synthase 1 (platelet, cytochrome P450, family 5, subfamily A) isoform TXS-II
EDARADD ENO1P protein
SELP P-selectin precursor
PTGS1 Cyclooxygenase 1b3
LTBP1 Latent-transforming growth factor beta-binding protein, isoform 1L precursor
Myosin-9
Hypothetical protein FLJ25678
Leukocyte elastase inhibitor

### Involved in Metabolic Processes:

Hypothetical protein FLJ25678
Phosphoglycerate kinase 1
Carbonic anhydrase 2
Peptidyl-prolyl cis-trans isomerase
SLC2A3 Solute carrier family 2, facilitated glucose transporter member 3
Isoform M1 of Pyruvate kinase isozymes M1/M2
L-lactate dehydrogenase B chain
Lactate dehydrogenase A
RcTPI1 (Fragment)

### Cytoskeletal:

Clathrin heavy chain 1
Myosin-9
Actin-like protein 3
Isoform 1 of Alpha-parvin
TPM 1 tropomyosin 1 alpha chain isoform 7
ARPC1B Actin-related protein ⅔ complex subunit 1B
COTL1 Coactosin-like protein
STXBP2 Syntaxin-binding protein 2
VASP Vasodilator-stimulated phosphoprotein
EHD1 EH domain-containing protein 1
CAPZA2 F-actin capping protein subunit alpha-2
ARPC4 Actin-related protein ⅔ complex subunit 4
FLNB Isoform 1 of Filamin-B
ARPC2 PNAS-139
SEPT11 Septin-11
ACTN4 Alpha-actinin-4
SPTBN5 Spectrin beta chain, brain 4
Beta-parvin
Alpha-actinin-1
271 kDa protein
Tubulin beta-2C chain
Transitional endoplasmic reticulum ATPase
Transgelin-2
Isoform 1 of Vinculin
Isoform 1 of Gelsolin precursor
Tubulin alpha-1 chain
Myosin regulatory light chain
Tropomyosin 4
Tubulin beta-1 chain
Myosin regulatory light polypeptide 9 isoform b
Cofilin-1
   Associated with Endoplasmic Reticulum:
Transitional endoplasmic reticulum ATPase
Hypothetical protein
Endoplasmin precursor
Isoform SERCA3B of Sarcoplasmic/endoplasmic reticulum calcium ATPase 3
CDNA FLJ45525 fis-clone BRTHA2026311-highly similar to Protein disulfide isomerase A6
CANX Calnexin precursor
RTN4 Isoform 1 of Reticulon-4
Peptidylprolyl isomerase B precursor

### Ion Pumps and Channels:

Isoform SERCA3B of Sarcoplasmic/endoplasmic reticulum calcium ATPase 3
KCNK15 Potassium channel subfamily K member 15
Chloride intracellular channel protein 1

### Involved in Angiogenesis:

Thrombospondin-2 precursor
Thrombospondin-1 precursor

### Small GTPases:

Ras-related protein Rab-27B
Ras-related protein Rab-11B
Rab GDP dissociation inhibitor alpha
RAB6B Ras-related protein Rab-6B
RAC2 Ras-related C3 botulinum toxin substrate 2 precursor
Rho GDP-dissociation inhibitor 2
Ras suppressor protein 1

### Mast Cell-Associated:

Ras-related protein Rab-27B
STXBP2 Syntaxin-binding protein 2

### Platelet-Associated:

Thrombospondin-1 precursor
271 kDa protein
Tubulin beta-1 chain
Coagulation factor XIII A chain precursor
TBXAS1 thromboxane A synthase 1 (platelet, cytochrome P450, family 5, subfamily A) isoform TXS-II
EDARADD ENO1P protein
PTGS1 Cyclooxygenase 1b3
Platelet glycoprotein V precursor
Pleckstrin
Multimerin-1 precursor
Isoform Beta-3B of Integrin beta-3 precursor

### Surface Receptors or Associated:

Thrombospondin-1 precursor
Platelet glycoprotein V precursor
Multimerin-1 precursor
Isoform Beta-3B of Integrin beta-3 precursor
SELP P-selectin precursor
SPARC SPARC precursor
Integrin-linked protein kinase 1
271 kDa protein

### Involved in Calcium Handling:

Isoform SERCA3B of Sarcoplasmic/endoplasmic reticulum calcium ATPase 3
CANX Calnexin precursor
Calpain-1 catalytic subunit

### Oxidative Stress:

Rab GDP dissociation inhibitor alpha
Protein DJ-1
Glutathione transferase omega-1
GPX1 glutathione peroxidase 1 isoform 1
SOD1 16 kDa protein
Glutathione S-transferase P
Superoxide dismutase [Mn]-mitochondrial precursor

### Mitochondrial Proteins:

Superoxide dismutase [Mn]-mitochondrial precursor
MRPS31 28S ribosomal protein S31, mitochondrial precursor
IDH2 Isocitrate dehydrogenase [NADP]-mitochondrial precursor

### Signaling Proteins:

Rab GDP dissociation inhibitor alpha
Integrin-linked protein kinase 1
Pleckstrin
Ras-related protein Rab-27B
Ras-related protein Rab-11B
RAB6B Ras-related protein Rab-6B
RAC2 Ras-related C3 botulinum toxin substrate 2 precursor
Rho GDP-dissociation inhibitor 2
Ras suppressor protein 1
VASP Vasodilator-stimulated phosphoprotein
EHD1 EH domain-containing protein 1
MAPRE2 Isoform 1 of Microtubule-associated protein RP/EB family member 2
Isoform 2 of Unc-112-related protein 2
Adenylyl cyclase-associated protein 1
PINCH protein
14-3-3 protein epsilon
14-3-3 protein eta
14-3-3 protein zeta/delta
14-3-3 protein gamma
Serum deprivation-response protein

### Nuclear Proteins and Transcription Factors:

MAPRE2 Isoform 1 of Microtubule-associated protein RP/EB family member 2
LYAR Cell growth-regulating nucleolar protein
EHD3 EH domain-containing protein 3
S100A4 Protein S100-A4
enolase 1
   Potentially Associated with Hemolysis:
ANK1 Isoform Er1 of Ankyrin-1
SPTA1 Spectrin alpha chain, erythrocyte
EPB41 Isoform 1 of Protein 4.1
Actin-like protein 2
Isoform 1 of Filamin-C
Filamin A, alpha
F-actin capping protein alpha-1 subunit
PDZ and LIM domain protein 1
Fructose-bisphosphate aldolase A
Band 3 anion transport protein
EPB42 Isoform Long of Erythrocyte membrane protein band 4.2
Isoform 1 of F-actin capping protein subunit beta
PKLR Isoform R-type of Pyruvate kinase isozymes R/L
   Proteins Underexpressed in Nicroparticles from AAA Patients:
19 kDa protein
ALB protein
ALB protein
Apolipoprotein A-I precursor
Apolipoprotein A-IV precursor
Apolipoprotein E precursor
apolipoprotein F precursor
C4b-binding protein alpha chain precursor
Carbonic anhydrase 1
Clusterin precursor
Complement C4-A precursor
Factor VII active site mutant immunoconjugate
FLJ00385 protein (Fragment)
Galectin-3-binding protein precursor
Hypothetical protein DKFZp686T04196 (Fragment)
Ig kappa chain V-III region HAH precursor
Ig mu heavy chain disease protein
IGHA1 protein
IGHM protein
IGKV1-5 protein
IGLC1 protein
Isoform 2 of Reelin precursor
PREDICTED: HEG homolog 1
PRO2275
Syntenin-1
Transferrin receptor protein 1
Vitronectin precursor
von Willebrand factor precursor

Table 1 provides a list of proteins found in all AAA cases used in this study, that were not found in controls. Note that the column entitled “Links” refers to the database identifier number and link number for the “Bioinformatic Harvester” gene and protein database and bioinformatic website supported by the Karlsruhe Institute of Technology, University of Karlsruhe, Germany. The sequences are available therein and are encompassed by the invention. Similar information for other protein biomarkers of the invention and their accession numbers is provided in Tables 2, 3, 4, 5, and 6.

TABLE 1
Proteins found in all AAA cases and no controls (2)

| Protein | Accession | p val | Links | |
|---|---|---|---|---|
| FHL1 Four and a half LIM | 14398 | 1.42792E−08 | IPI00014398 | LIM domain, a conserved cysteine-rich |
| domains 1 variant | | | | module present in more than 100 different |
| | | | | human proteins. The human four-and-a |
| | | | | half-LIM-only protein family consists of the |
| | | | | members FHL1, FHL2, FHL3, FHL4 and |
| | | | | ACT. Associated with hemophagocytic |
| | | | | lymphohistiocytosis. |
| COTL1 Coactosin-like protein | 17704 | 4.37894E−05 | IPI00017704 | binds F-actin, and also interacts with 5- |
| | | | | lipoxygenase, which is the first committed |
| | | | | enzyme in leukotriene biosynthesis. |
| | | | | coactosin-like protein (CLP) is a small |
| | | | | (MW approximately 17 kDa) evolutionarily |
| | | | | conserved actin-binding protein. It can bind |
| | | | | to actin filaments but not globular actin and |
| | | | | belongs to the fourth class of ADF-H- |
| | | | | domain-containing proteins |

TABLE 2
Proteins found in all AAA cases and some controls (47)

| Protein | Accession | p val | Links | |
|---|---|---|---|---|
| Integrin-linked | 13219 | 9.92596E−11 | IPI00013219 | Integrin-linked kinase (ILK), interacts with the |
| protein kinase 1 | | | | cytoplasmic domain of beta-1 integrin. |
| peptidylprolyl | 646304 | 1.32589E−10 | IPI00646304 | Cyclophilin-B is a cyclosporine-binding protein and is |
| isomerase B | | | | mainly located within the endoplasmic reticulum. It is |
| precursor | | | | associated with the secretory pathway and released in |
| | | | | biological fluids |
| Protein disulfide- | 10796 | 1.95505E−09 | IPI00010796 | ECM-related. prolyl 4-hydroxylase, a highly abundant |
| isomerase | | | | multifunctional enzyme that belongs to the protein |
| precursor | | | | disulfide isomerase family. When present as a tetramer |
| | | | | consisting of two alpha and two beta subunits, this enzyme |
| | | | | is involved in hydroxylation of prolyl residues in |
| | | | | preprocollagen |
| Glyceraldehyde- | 219018 | 2.45372E−09 | IPI00219018 | Housekeeping enzyme (cell death?) Glyceraldehyde-3- |
| 3-phosphate | | | | phosphate dehydrogenase catalyzes an important energy- |
| dehydrogenase | | | | yielding step in carbohydrate metabolism |
| Beta-parvin | 43083 | 4.47811E−09 | IPI00043083 | PARVB is an actin-binding proteins associated with focal |
| | | | | contacts |
| RAC2 Ras- | 10270 | 4.65238E−09 | IPI00010270 | central small GTPase. Every cell has it. |
| related C3 | | | | |
| botulinum toxin | | | | |
| substrate 2 | | | | |
| precursor | | | | |
| Isoform 2 of | 216699 | 6.98532E−09 | IPI00216699 | (This is URP2. The closest: URP1: a member of a novel |
| Unc-112-related | | | | family of PH and FERM domain-containing membrane- |
| protein 2 | | | | associated proteins is significantly over-expressed in lung |
| | | | | and colon carcinomas |
| Isoform M1 of | 220644 | 2.03687E−08 | IPI00220644 | pyruvate kinase that catalyzes the production of |
| Pyruvate kinase | | | | phosphoenolpyruvate from pyruvate and ATP |
| isozymes M1/M2 | | | | |
| L-lactate | 219217 | 6.48936E−08 | IPI00219217 | LDHB and peptidase B (169900) are linked on |
| dehydrogenase B | | | | chromosome 12 LDHB deficiency in serum, saliva and |
| chain | | | | erythrocytes of a 64-year-old male with mild diabetes. |
| | | | | Correlated with lung, prostate cancer, but these may |
| | | | | reflect loss of short arm of chr 12. |
| Alpha-actinin-1 | 13508 | 2.12222E−07 | IPI00013508 | Alpha actinin, very common cytoskeletal protein, |
| | | | | homodimer of 97-kD subunits arranged in antiparallel |
| | | | | fashion. In myofibrillar cells, alpha-actinin constitutes a |
| | | | | major component of Z-discs in striated muscle and of the |
| | | | | functionally analogous dense bodies and dense plaques in |
| | | | | smooth muscle. In nonmuscle cells, it is distributed along |
| | | | | microfilament bundles and is thought to mediate their |
| | | | | attachment to the membrane at adherens-type junctions |
| 271 kDa protein | 298994 | 3.35797E−07 | IPI00298994 | Talin is found in cell-substratum and cell-cell contacts. |
| | | | | This protein plays a significant role in the assembly of |
| | | | | actin filaments and in spreading and migration of various |
| | | | | cell types, including fibroblasts and osteoclasts. It |
| | | | | codistributes with integrins in the cell surface membrane |
| | | | | in order to assist in the attachment of adherent cells to |
| | | | | extracellular matrices. Very common in platelets |
| Pleckstrin | 306311 | 3.47376E−07 | IPI00306311 | Pleckstrin. In platelets, agonists that stimulate |
| | | | | phosphoinositide turnover cause the rapid phosphorylation |
| | | | | of a protein of apparent relative molecular mass 40,000-47,000, |
| | | | | called P47, by protein kinase C. Mutations in this |
| | | | | gene have been associated with Loeys-Dietz aortic |
| | | | | aneurysm syndrome (LDAS) |
| Endoplasmin | 27230 | 3.56368E−07 | IPI00027230 | endoplasmin or GRP94 (heat shock protein 90 family). |
| precursor | | | | Involved in misfolded protein response, which occurs in |
| | | | | foam cells. Not much known! |
| Thrombospondin- | 296099 | 4.8984E−07 | IPI00296099 | Thrombospondin-1. Adhesive glycoprotein that mediates |
| 1 precursor | | | | cell-to-cell and cell-to-matrix interactions. This protein |
| | | | | can bind to fibrinogen, fibronectin, laminin, type V |
| | | | | collagen and integrins alpha-V/beta-1. This protein has |
| | | | | been shown to play roles in platelet aggregation, |
| | | | | angiogenesis, and tumorigenesis. |
| Adenylyl | 8274 | 5.3996E−07 | IPI00008274 | CAP, adenylate cyclase-associated protein 1; cyclic AMP |
| cyclase- | | | | pathway. |
| associated protein 1 | | | | |
| PINCH protein | 7634 | 7.44591E−07 | IPI00007634 | adaptor protein which contains five LIM domains, or |
| | | | | double zinc fingers. The protein is likely involved in |
| | | | | integrin signaling through its LIM domain-mediated |
| | | | | interaction with integrin-linked kinase, found in focal |
| | | | | adhesion plaques. It is also thought to act as a bridge |
| | | | | linking integrin-linked kinase to NCK adaptor protein 2, |
| | | | | which is involved in growth factor receptor kinase |
| | | | | signaling pathways. |
| lactate | 217966 | 1.23774E−06 | IPI00217966 | Indicates (smooth) muscle death. Lactate dehydrogenase A |
| dehydrogenase A | | | | catalyzes the conversion of L-lactate and NAD to pyruvate |
| | | | | and NADH in the final step of anaerobic glycolysis. |
| | | | | LDHA is found predominantly in muscle tissue |
| RcTPI1 | 383071 | 7.4725E−06 | IPI00383071 | Triosephosphate isomerase. Practically nothing known. |
| (Fragment) | | | | |
| Multimerin-1 | 12269 | 9.2125E−06 | IPI00012269 | platelets and in the endothelium. Multimerin is a factor |
| precursor | | | | V/Va-binding protein and may function as a carrier protein |
| | | | | for platelet factor V. autosomal-dominant bleeding |
| | | | | disorder (factor V Quebec) were found to have a |
| | | | | deficiency of platelet multimerin. This is the first one |
| | | | | that is not RBC-related. |
| Glutathione S- | 219757 | 1.54844E−05 | IPI00219757 | Glutathione S-transferases (GSTs) are a family of enzymes |
| transferase P | | | | that play an important role in detoxification. glutathione S- |
| | | | | transferase pi gene (GSTP1) is a polymorphic gene |
| | | | | encoding active, functionally different GSTP1 variant |
| | | | | proteins that are thought to function in xenobiotic |
| | | | | metabolism |
| Isoform Beta-3B | 220350 | 1.5803E−05 | IPI00220350 | integrin, beta 3. this is probably platelet Iib/IIIa. Klaus' |
| of Integrin beta-3 | | | | area of expertise |
| precursor | | | | |
| Myosin-9 | 19502 | 1.70202E−05 | IPI00019502 | This is in the uropod of leukocytes. Specifically reacts |
| | | | | with a 224-kD polypeptide in leukocyte cell lines, and the |
| | | | | protein was upregulated during the induction of monocytic |
| | | | | and granulocytic differentiation in these cells. |
| Tubulin beta-2C | 7752 | 1.90208E−05 | IPI00007752 | Tubulin beta2C, nothing known |
| chain | | | | |
| Coagulation | 297550 | 1.97992E−05 | IPI00297550 | Coagulation factor XIII is the last zymogen to become |
| factor XIII A | | | | activated in the blood coagulation cascade. Plasma factor |
| chain precursor | | | | XIII is a heterotetramer composed of 2 A subunits and 2 B |
| | | | | subunits. The A subunits have catalytic function, and the |
| | | | | B subunits do not have enzymatic activity and may serve |
| | | | | as plasma carrier molecules. |
| 14-3-3 protein | 816 | 2.1074E−05 | IPI00000816 | Tyrosine 3-monooxygenase/tryptophan 5-monooxygenase |
| epsilon | | | | activation protein, epsilon polypeptide, belongs to the 14- |
| | | | | 3-3 family of proteins which mediate signal transduction |
| | | | | by binding to phosphoserine-containing proteins. Could be |
| | | | | a caspase. |
| Transitional | 22774 | 3.21226E−05 | IPI00022774 | Valosin-containing protein (VCP) is a member of a family |
| endoplasmic | | | | that includes putative ATP-binding proteins involved in |
| reticulum | | | | vesicle transport and fusion, 26S proteasome function, and |
| ATPase | | | | assembly of peroxisomes. VCP, as a structural protein, is |
| | | | | associated with clathrin, and heat-shock protein Hsc70, to |
| | | | | form a complex. |
| Hypothetical | 17672 | 3.37229E−05 | IPI00017672 | NP encodes the enzyme purine nucleoside phosphorylase |
| protein FLJ25678 | | | | that together with adenosine deaminase (ADA) serves a |
| | | | | key role in purine catabolism, referred to as the salvage |
| | | | | pathway. Mutations in either enzyme result in a severe |
| | | | | combined immunodeficiency (SCID). |
| Transgelin-2 | 550363 | 3.83393E−05 | IPI00550363 | Transgelin is one of the earliest markers of differentiated |
| | | | | smooth muscle. |
| Isoform 1 of | 291175 | 6.07916E−05 | IPI00291175 | Vinculin is a cytoskeletal protein associated with cell-cell |
| Vinculin | | | | and cell-matrix junctions, where it is thought to function |
| | | | | as one of several interacting proteins involved in |
| | | | | anchoring F-actin to the membrane. |
| Isoform 1 of | 26314 | 6.69516E−05 | IPI0026314 | Gelsolin |
| Gelsolin | | | | |
| precursor | | | | |
| 14-3-3 protein eta | 216319 | 7.22044E−05 | IPI00216319 | tyrosine 3-monooxygenase/tryptophan 5-monooxygenase |
| | | | | activation protein; 14-3-3 family of proteins which |
| | | | | mediate signal transduction by binding to phosphoserine- |
| | | | | containing proteins. This highly conserved protein family |
| | | | | is found in both plants and mammals, and this protein is |
| | | | | 99% identical to the mouse, rat and bovine orthologs. |
| Tubulin alpha-1 | 7750 | 7.83634E−05 | IPI00007750 | alpha, beta and gamma tubulin families are found in all |
| chain | | | | eukaryotes. The alpha and beta tubulins represent the |
| | | | | major components of microtubules, while gamma tubulin |
| | | | | plays a critical role in the nucleation of microtubule |
| | | | | assembly. There are multiple alpha and beta tubulin genes |
| | | | | and they are highly conserved among and between species. |
| | | | | This gene encodes an alpha tubulin that is a highly |
| | | | | conserved homolog of a rat testis-specific alpha tubulin. |
| Isoform 1 of Heat | 3865 | 9.84562E−05 | IPI00003865 | heat shock protein 70 family. Associated with protein |
| shock cognate 71 kDa | | | | folding. |
| protein | | | | |
| Superoxide | 22314 | 0.000105652 | IPI00022314 | iron/manganese superoxide dismutase family. It encodes a |
| dismutase [Mn], | | | | mitochondrial protein that forms a homotetramer and |
| mitochondrial | | | | binds one manganese ion per subunit. This protein binds to |
| precursor | | | | the superoxide byproducts of oxidative phosphorylation |
| | | | | and converts them to hydrogen peroxide and diatomic |
| | | | | oxygen. |
| Chloride | 10896 | 0.000162753 | IPI00010896 | Stabilization of cell membrane potential, transepithelial |
| intracellular | | | | transport, maintenance of intracellular pH, and regulation |
| channel protein 1 | | | | of cell volume. Chloride intracellular channel 1 is a |
| | | | | member of the p64 family; the protein localizes principally |
| | | | | to the cell nucleus and exhibits both nuclear and plasma |
| | | | | membrane chloride ion channel activity. |
| Rho GDP- | 3817 | 0.000180755 | IPI00003817 | apoptosis-related, interacts with vav1, could be uropod |
| dissociation | | | | |
| inhibitor 2 | | | | |
| Myosin | 33494 | 0.000206338 | IPI00033494 | The activity of nonmuscle myosin II (see MYH9; MIM |
| regulatory light | | | | 160775) is regulated by phosphorylation of a regulatory |
| chain | | | | light chain, such as MRLC2. This phosphorylation results |
| | | | | in higher MgATPase activity and the assembly of myosin |
| | | | | II filaments. Might be integrin-related |
| enolase 1 | 465248 | 0.000208539 | IPI00465248 | Strange biology: encodes alpha-enolase, a homodimeric |
| | | | | soluble enzyme, and also encodes a shorter monomeric |
| | | | | structural lens protein, tau-crystallin. The two proteins are |
| | | | | made from the same message. The full length protein, the |
| | | | | isoenzyme, is found in the cytoplasm. The shorter protein |
| | | | | is produced from an alternative translation start, is |
| | | | | localized to the nucleus, and has been found to bind to an |
| | | | | element in the c-myc promoter. |
| Leukocyte | 27444 | 0.000676001 | IPI00027444 | dual specificity inhibitor with two adjacent reactive sites |
| elastase inhibitor | | | | that support rapid efficient inhibitory reactions with |
| | | | | cellular proteases, including the three neutrophil granule |
| | | | | proteases |
| 14-3-3 protein | 21263 | 0.000849047 | IPI00021263 | 14-3-3 family of proteins which mediate signal |
| zeta/delta | | | | transduction by binding to phosphoserine-containing |
| | | | | proteins. This highly conserved protein family is 99% |
| | | | | identical to the mouse, rat and sheep orthologs. The |
| | | | | encoded protein interacts with IRS1 protein: insulin |
| | | | | signaling |
| Tropomyosin 4 | 10779 | 0.000890756 | IPI00010779 | nothing known. May be expressed in muscle and CD34+ |
| | | | | fibroblasts |
| Tubulin beta-1 | 6510 | 0.001345954 | IPI00006510 | Platelet protein. Q43P reduces cardiovascular risk. |
| chain | | | | Association of the beta1-tubulin Q43P polymorphism with |
| | | | | intracerebral hemorrhage in men |
| myosin | 30929 | 0.002206947 | IPI00030929 | Myosin light chain that may regulate muscle contraction |
| regulatory light | | | | by modulating the ATPase activity of myosin heads. The |
| polypeptide 9 | | | | encoded protein binds calcium and is activated by myosin |
| isoform b | | | | light chain kinase. |
| Ras suppressor | 17256 | 0.002565458 | IPI00017256 | Ras signal transduction pathway, growth inhibition, and |
| protein 1 | | | | nerve-growth factor induced differentiation processes. The |
| | | | | Ras suppressor Rsu-1 binds to the LIM 5 domain of the |
| | | | | adaptor protein PINCH1 and participates in adhesion- |
| | | | | related functions. |
| Cofilin-1 | 12011 | 0.002783117 | IPI0002011 | Anti-apoptotic. Caspase-11 regulates cell migration by |
| | | | | promoting Aip1-Cofilin-mediated actin depolymerization. |
| | | | | Cofilin is widely distributed intracellular actin-modulating |
| | | | | protein that binds and depolymerizes filamentous F-actin |
| | | | | and inhibits the polymerization of monomeric G-actin in a |
| | | | | pH-dependent manner. It is involved in the translocation |
| | | | | of actin-cofilin complex from cytoplasm to nucleus. |
| 14-3-3 protein | 220642 | 0.003545662 | IPI00220642 | 14-3-3 family of proteins which mediate signal |
| gamma | | | | transduction by binding to phosphoserine-containing |
| | | | | proteins. This protein is 100% identical to the rat ortholog. |
| | | | | It is induced by growth factors in human vascular smooth |
| | | | | muscle cells, and is also highly expressed in skeletal and |
| | | | | heart muscles |
| Serum | 5809 | 0.005995663 | IPI00005809 | calcium-independent phospholipid-binding protein whose |
| deprivation- | | | | expression increases in serum-starved cells. This protein |
| response protein | | | | has also been shown to be a substrate for protein kinase C |
| | | | | (PKC) phosphorylation. |

TABLE 3
Proteins found in most AAA cases and no controls (32)

| Protein | Accession | p val | Links | |
|---|---|---|---|---|
| IDH2 Isocitrate dehydrogenase | 11107 | 5.2062E−07 | IPI00011107 | NADP(+)-dependent isocitrate |
| [NADP]-mitochondrial | | | | dehydrogenase found in the mitochondria. |
| precursor | | | | It plays a role in intermediary metabolism |
| | | | | and energy production. This protein may |
| | | | | tightly associate or interact with the |
| | | | | pyruvate dehydrogenase complex. |
| STXBP2 Syntaxin-binding | 19971 | 2.63074E−06 | IPI00019971 | regulates exocytotic membrane fusion |
| protein 2 | | | | positively interacting with syntaxin-3, in |
| | | | | mast cells |
| CANX Calnexin precursor | 20984 | 9.8479E−06 | IPI00020984 | calcium-binding, endoplasmic reticulum |
| | | | | (ER)-associated protein that interacts |
| | | | | transiently with newly synthesized N-linked |
| | | | | glycoproteins, facilitating protein folding |
| | | | | and assembly. It may also play a central |
| | | | | role in the quality control of protein folding |
| | | | | by retaining incorrectly folded protein |
| | | | | subunits within the ER for degradation. |
| | | | | Really nothing known. ER stress? |
| GPX1 glutathione peroxidase 1 | 293975 | 1.11268E−05 | IPI00293975 | detoxification of hydrogen peroxide, and is |
| isoform 1 | | | | one of the most important antioxidant |
| | | | | enzymes in humans. It has been reported |
| | | | | that the protein encoded by this gene |
| | | | | protects from CD95-induced apoptosis in |
| | | | | cultured breast cancer cells and inhibits 5- |
| | | | | lipoxygenase in blood cells, and its |
| | | | | overexpression delays endothelial cell |
| | | | | growth and increases resistance to toxic |
| | | | | challenges. No Abstract Glutathione |
| | | | | peroxidase in human and animal aortas. |
| VASP Vasodilator-stimulated | 301058 | 1.42477E−05 | IPI00301058 | Platelet protein, member of the Ena-VASP |
| phosphoprotein | | | | protein family. Ena-VASP family members |
| | | | | contain an EHV1 N-terminal domain that |
| | | | | binds proteins containing E/DFPPPPXD/E |
| | | | | motifs and targets Ena-VASP proteins to |
| | | | | focal adhesions. In the mid-region of the |
| | | | | protein, family members have a proline-rich |
| | | | | domain that binds SH3 and WW domain- |
| | | | | containing proteins. Their C-terminal |
| | | | | EVH2 domain mediates tetramerization and |
| | | | | binds both G and F actin. VASP is |
| | | | | associated with filamentous actin formation |
| | | | | and likely plays a widespread role in cell |
| | | | | adhesion and motility. VASP may also be |
| | | | | involved in the intracellular signaling |
| | | | | pathways that regulate integrin-extracellular |
| | | | | matrix interactions. VASP is regulated by |
| | | | | the cyclic nucleotide-dependent kinases |
| | | | | PKA and PKG. |
| SPARC SPARC precursor | 14572 | 1.43198E−05 | IPI00014572 | Secreted protein acidic and rich in |
| | | | | cysteine/osteonectin/BM40, or SPARC, is a |
| | | | | matrix-associated protein that elicits |
| | | | | changes in cell shape, inhibits cell-cycle |
| | | | | progression, and influences the synthesis of |
| | | | | extracellular matrix (ECM). May bind |
| | | | | VCAM-1 (Kelly) |
| NME1 Nucleoside diphosphate | 12048 | 4.27395E−05 | IPI00012048 | Reduced mRNA transcript levels in highly |
| kinase A | | | | metastatic cells. Nucleoside diphosphate |
| | | | | kinase (NDK) exists as a hexamer |
| | | | | composed of ‘A’ (encoded by this gene) and |
| | | | | ‘B’ (encoded by NME2) isoforms. |
| | | | | Mutations in this gene have been identified |
| | | | | in aggressive neuroblastomas. |
| EHD1 EH domain-containing | 17184 | 5.65707E−05 | IPI00017184 | highly conserved gene family encoding |
| protein 1 | | | | EPS15 homology (EH) domain-containing |
| | | | | proteins. The protein-binding EH domain |
| | | | | was first noted in EPS15, a substrate for the |
| | | | | epidermal growth factor receptor. The EH |
| | | | | domain has been shown to be an important |
| | | | | motif in proteins involved in protein- |
| | | | | protein interactions and in intracellular |
| | | | | sorting. The protein encoded by this gene is |
| | | | | thought to play a role in the endocytosis of |
| | | | | IGF1 receptors. |
| HSPC159 Galectin-related | 23549 | 7.18221E−05 | IPI00023549 | Lens protein, also found in primary |
| protein | | | | testicular cancer and its lung metastases |
| LYAR Cell growth-regulating | 15838 | 8.35279E−05 | IPI00015838 | a novel nucleolar protein with zinc finger |
| nucleolar protein | | | | DNA-binding motifs, is involved in cell |
| | | | | growth regulation |
| CAPZA2 F-actin capping | 26182 | 0.000304865 | IPI00026182 | member of the F-actin capping protein |
| protein subunit alpha-2 | | | | alpha subunit family. It is the alpha subunit |
| | | | | of the barbed-end actin binding protein Cap |
| | | | | Z. By capping the barbed end of actin |
| | | | | filaments, Cap Z regulates the growth of the |
| | | | | actin filaments at the barbed end. |
| SLC2A3 Solute carrier family | 3909 | 0.000645755 | IPI00003909 | GLUT3 as the brain-type glucose |
| 2, facilitated glucose | | | | transporter. It appears that high GLUT3 |
| transporter member 3 | | | | protein expression is confined generally to |
| | | | | tissues that exhibit a high glucose demand, |
| | | | | such as brain and nerve. GLUT3 mRNA |
| | | | | was present in the trophoblast cell layer and |
| | | | | in vascular endothelium with a |
| | | | | heterogeneous distribution pattern. |
| RAB6B Ras-related protein | 16891 | 0.000682546 | IPI00016891 | Family of small GTPases consists of three |
| Rab-6B | | | | different isoforms: Rab6A, Rab6A′ and |
| | | | | Rab6B. Both Rab6A and Rab6A′ are |
| | | | | ubiquitously expressed whereas Rab6B is |
| | | | | predominantly expressed in brain. Recent |
| | | | | studies have shown that Rab6A′ is the |
| | | | | isoform regulating the retrograde transport |
| | | | | from late endosomes via the Golgi to the |
| | | | | ER and in the transition from anaphase to |
| | | | | metaphase during mitosis. ER stress? |
| EHD3 EH domain-containing | 21458 | 0.001186345 | IPI00021458 | Contains nucleotide-binding consensus site |
| protein 3 | | | | at the N terminus, a bipartite nuclear |
| | | | | localization signal, and an eps15 homology |
| | | | | (EH) protein-binding domain with an EF- |
| | | | | hand motif at the C terminus. EHD3 |
| | | | | encodes a deduced 546-amino acid protein. |
| | | | | Northern blot analysis detected a 3.6-kb |
| | | | | EHD3 transcript that is highly expressed in |
| | | | | heart and brain and moderately expressed in |
| | | | | kidney, liver, and placenta. |
| SOD1 16 kDa protein | 218733 | 0.001625909 | IPI00218733 | one of two isozymes responsible for |
| | | | | destroying free superoxide radicals in the |
| | | | | body. The encoded isozyme is a soluble |
| | | | | cytoplasmic protein, acting as a homodimer |
| | | | | to convert naturally-occurring but harmful |
| | | | | superoxide radicals to molecular oxygen |
| | | | | and hydrogen peroxide. The other isozyme |
| | | | | is a mitochondrial protein. Mutations in this |
| | | | | gene have been implicated as causes of |
| | | | | familial amyotrophic lateral sclerosis. |
| ARPC4 Actin-related protein | 554811 | 0.001647049 | IPI00554811 | one of seven subunits of the human Arp2/3 |
| 2/3 complex subunit 4 | | | | protein complex. The Arp2/3 protein |
| | | | | complex has been implicated in the control |
| | | | | of actin polymerization in cells and has |
| | | | | been conserved through evolution. The |
| | | | | exact role of the protein encoded by this |
| | | | | gene, the p20 subunit, has yet to be |
| | | | | determined. |
| FLNB Isoform 1 of Filamin-B | 289334 | 0.001752632 | IPI00289334 | contains an N-terminal actin-binding |
| | | | | domain, a backbone of 24 tandem repeats, |
| | | | | and 2 hinge regions. Excluding the unique |
| | | | | first hinge region of beta-filamin, the |
| | | | | sequences of beta-filamin and ABP280 are |
| | | | | 70% identical. Antibodies against beta- |
| | | | | filamin detected a 280-kD protein on |
| | | | | Western blots of human umbilical vein |
| | | | | endothelial cell (HUVEC) extracts and |
| | | | | stained normal human endothelial cells in |
| | | | | culture and in situ. |
| TBXAS1 thromboxane A | 8967 | 0.001789255 | IPI00008967 | member of the cytochrome P450 |
| synthase 1 (platelet, | | | | superfamily of enzymes. The cytochrome |
| cytochrome P450, family 5, | | | | P450 proteins are monooxygenases which |
| subfamily A) isoform TXS-II | | | | catalyze many reactions involved in drug |
| | | | | metabolism and synthesis of cholesterol, |
| | | | | steroids and other lipids. However, this |
| | | | | protein is considered a member of the |
| | | | | cytochrome P450 superfamily on the basis |
| | | | | of sequence similarity rather than |
| | | | | functional similarity. This endoplasmic |
| | | | | reticulum membrane protein catalyzes the |
| | | | | conversion of prostaglandin H2 to |
| | | | | thromboxane A2, a potent vasoconstrictor |
| | | | | and inducer of platelet aggregation. The |
| | | | | enzyme plays a role in several |
| | | | | pathophysiological processes including |
| | | | | hemostasis, cardiovascular disease, and |
| | | | | stroke. |
| EDARADD ENO1P protein | 328587 | 0.002329786 | IPI00328587 | member of the cytochrome P450 |
| | | | | superfamily of enzymes. The cytochrome |
| | | | | P450 proteins are monooxygenases which |
| | | | | catalyze many reactions involved in drug |
| | | | | metabolism and synthesis of cholesterol, |
| | | | | steroids and other lipids. However, this |
| | | | | protein is considered a member of the |
| | | | | cytochrome P450 superfamily on the basis |
| | | | | of sequence similarity rather than |
| | | | | functional similarity. This endoplasmic |
| | | | | reticulum membrane protein catalyzes the |
| | | | | conversion of prostaglandin H2 to |
| | | | | thromboxane A2, a potent vasoconstrictor |
| | | | | and inducer of platelet aggregation. The |
| | | | | enzyme plays a role in several |
| | | | | pathophysiological processes including |
| | | | | hemostasis, cardiovascular disease, and |
| | | | | stroke. |
| RTN4 Isoform 1 of Reticulon-4 | 21766 | 0.002409873 | IPI00021766 | Reticulons are associated with the |
| | | | | endoplasmic reticulum, and are involved in |
| | | | | neuroendocrine secretion or in membrane |
| | | | | trafficking in neuroendocrine cells. The |
| | | | | product of this gene is a potent neurite |
| | | | | outgrowth inhibitor which may also help |
| | | | | block the regeneration of the central |
| | | | | nervous system in higher vertebrates. ER |
| | | | | stress? |
| SELP P-selectin precursor | 295339 | 0.002662904 | IPI00295339 | This is P-selectin. Attenuation of |
| | | | | experimental aortic aneurysm formation in |
| | | | | P-selectin knockout mice. |
| S100A4 Protein S100-A4 | 32313 | 0.002792494 | IPI00032313 | member of the S100 family of proteins |
| | | | | containing 2 EF-hand calcium-binding |
| | | | | motifs. S100 proteins are localized in the |
| | | | | cytoplasm and/or nucleus of a wide range |
| | | | | of cells, and involved in the regulation of a |
| | | | | number of cellular processes such as cell |
| | | | | cycle progression and differentiation. |
| PTGS1 Cyclooxygenase 1b3 | 298267 | 0.003734347 | IPI00298267 | This is COX1 |
| KCNK15 Potassium channel | 7134 | 0.003852598 | IPI00007134 | superfamily of potassium channel proteins |
| subfamily K member 15 | | | | containing two pore-forming P domains. |
| | | | | The product of this gene has not been |
| | | | | shown to be a functional channel, however, |
| | | | | it may require other non-pore-forming |
| | | | | proteins for activity. |
| LTBP1 Latent-transforming | 220249 | 0.0054602 | IPI00220249 | latent TGF-beta binding proteins (LTBPs). |
| growth factor beta-binding | | | | The secretion and activation of TGF-betas |
| protein, isoform 1L precursor | | | | is regulated by their association with |
| | | | | latency-associated proteins and with latent |
| | | | | TGF-beta binding proteins. The product of |
| | | | | this gene targets latent complexes of |
| | | | | transforming growth factor beta to the |
| | | | | extracellular matrix, where the latent |
| | | | | cytokine is subsequently activated by |
| | | | | several different mechanisms. |
| ARPC2 PNAS-139 | 477 | 0.006028532 | IPI00000477 | one of seven subunits of the human Arp2/3 |
| | | | | protein complex. The Arp2/3 protein |
| | | | | complex has been implicated in the control |
| | | | | of actin polymerization in cells and has |
| | | | | been conserved through evolution. The |
| | | | | exact role of the protein encoded by this |
| | | | | gene, the p34 subunit, has yet to be |
| | | | | determined. |
| MAPRE2 Isoform 1 of | 3420 | 0.006710365 | IPI00003420 | significant homology to the adenomatous |
| Microtubule-associated protein | | | | polyposis coli (APC) protein-binding EB1 |
| RP/EB family member 2 | | | | gene family. The function of this protein is |
| | | | | unknown; however, its homology suggests |
| | | | | involvement in tumorigenesis of colorectal |
| | | | | cancers and proliferative control of normal |
| | | | | cells. This gene may belong to the |
| | | | | intermediate/early gene family, involved in |
| | | | | the signal transduction cascade downstream |
| | | | | of the TCR. |
| SEPT11 Septin-11 | 19376 | 0.006736113 | IPI00019376 | Sept7/9b/11 form a complex that has effects |
| | | | | on filament elongation, bundling, or |
| | | | | disruption |
| TPI1 Isoform 2 of | 451401 | 0.007099212 | IPI00451401 | phosphorylated cofilin-triosephosphate |
| Triosephosphate isomerase | | | | isomerase (TPI) complex interacts with |
| | | | | Na, K-ATPase and enhances the pump |
| | | | | activity through the phosphorylation of |
| | | | | cofilin via Rho-mediated signaling |
| | | | | pathway. |
| ACTN4 Alpha-actinin-4 | 13808 | 0.007189079 | IPI00013808 | Alpha actinin is an actin-binding protein |
| | | | | with multiple roles in different cell types. In |
| | | | | nonmuscle cells, the cytoskeletal isoform is |
| | | | | found along microfilament bundles and |
| | | | | adherens-type junctions, where it is |
| | | | | involved in binding actin to the membrane. |
| | | | | In contrast, skeletal, cardiac, and smooth |
| | | | | muscle isoforms are localized to the Z-disc |
| | | | | and analogous dense bodies, where they |
| | | | | help anchor the myofibrillar actin filaments. |
| HSPA4 Heat shock 70 kDa | 2966 | 0.007280476 | IPI00002966 | This is hsp70. human heat-shock protein |
| protein 4 | | | | (hsp), which they designated hsp70 RY. |
| | | | | The cDNA was isolated from an EBV- |
| | | | | transformed B-cell line from a patient with |
| | | | | leukocyte adhesion molecule deficiency. |
| | | | | The predicted 701-amino acid protein has |
| | | | | an N-terminal ATP-binding domain and a |
| | | | | C-terminal peptide-binding domain |
| | | | | characteristic of hsp proteins. |
| SPTBN5 Spectrin beta chain, | 219168 | 0.00744737 | IPI00219168 | This is NOT erythrocyte spectrin. SPTBN5 |
| brain 4 | | | | has a modestly conserved actin-binding |
| | | | | domain, a conserved membrane-association |
| | | | | domain-1, a conserved self-association |
| | | | | domain, and a C-terminal pleckstrin |
| | | | | homology domain. The putative ankyrin- |
| | | | | binding domain of SPTBN5 is poorly |
| | | | | conserved and may be inactive. Based on |
| | | | | its structural features, Stabach and Morrow |
| | | | | (2000) proposed that SPTBN5 is likely to |
| | | | | form heterodimers and oligomers with |
| | | | | alpha-spectrin and to interact directly with |
| | | | | cellular membranes. Northern dot blot |
| | | | | analysis revealed SPTBN5 expression at |
| | | | | very low levels in many human tissues, |
| | | | | with strongest expression in cerebellum, |
| | | | | spinal cord, stomach, pituitary gland, liver, |
| | | | | pancreas, salivary gland, kidney, bladder, |
| | | | | and heart. |

TABLE 4
Proteins found in most AAA cases and some controls (25):

| Protein | Accession | p val | Links | |
|---|---|---|---|---|
| Hypothetical protein | 334775 | 2.11566E−05 | IPI00334775 | hsp90-B, not much known. Anti-heat shock |
| DKFZp761K0511 | | | | protein 90beta antibodies are detected in patients |
| | | | | with multiple sclerosis during remission. ER |
| | | | | stress, apoptosis. |
| Heat shock protein 86 | 31523 | 8.52482E−05 | IPI00031523 | The hsp90 alpha isoform, but not hsp90 beta, is |
| (Fragment) | | | | expressed extracellularly where it interacts with |
| | | | | the matrix metalloproteinase 2 (MMP2). |
| | | | | Inhibition of extracellular hsp90 alpha decreases |
| | | | | both MMP2 activity and invasiveness. |
| Phosphoglycerate kinase 1 | 169383 | 9.2557E−05 | IPI00169383 | The PGK1 gene encodes phosphoglycerate |
| | | | | kinase-1, also known as ATP: 3-phosphoglycerate |
| | | | | 1-phosphotransferase (EC 2.7.2.3), which |
| | | | | catalyzes the reversible conversion of 1,3- |
| | | | | diphosphoglycerate to 3-phosphoglycerate during |
| | | | | glycolysis, generating one molecule of ATP. |
| Actin-like protein 3 | 28091 | 0.000117359 | IPI00028091 | The specific function of this gene has not yet |
| | | | | been determined; however, the protein it encodes |
| | | | | is known to be a major constituent of the ARP2/3 |
| | | | | complex. |
| Isoform SERCA3B of | 4092 | 0.000180037 | IPI00004092 | SERCA Ca(2+)-ATPases, which are intracellular |
| Sarcoplasmic/endoplasmic | | | | pumps located in the sarcoplasmic or |
| reticulum calcium ATPase 3 | | | | endoplasmic reticula of muscle cells. This |
| | | | | enzyme catalyzes the hydrolysis of ATP coupled |
| | | | | with the translocation of calcium from the cytosol |
| | | | | to the sarcoplasmic reticulum lumen, and is |
| | | | | involved in calcium sequestration associated with |
| | | | | muscular excitation and contraction. Alternative |
| | | | | splicing results in multiple transcript variants |
| | | | | encoding different isoforms. |
| Thrombospondin-2 | 18769 | 0.000268125 | IPI00018769 | Disulfide-linked homotrimeric glycoprotein that |
| precursor | | | | mediates cell-to-cell and cell-to-matrix |
| | | | | interactions. This protein has been shown to |
| | | | | function as a potent inhibitor of tumor growth and |
| | | | | angiogenesis. Studies of the mouse counterpart |
| | | | | suggest that this protein may modulate the cell |
| | | | | surface properties of mesenchymal cells and be |
| | | | | involved in cell adhesion and migration. |
| Ras-related protein Rab- | 10491 | 0.000275128 | IPI00010491 | Rab27b regulates mast cell granule dynamics and |
| 27B | | | | secretion. |
| Carbonic anhydrase 2 | 218414 | 0.000414558 | IPI00218414 | CA2 is one of several (at least 7) isozymes of |
| | | | | carbonic anhydrase. Carbonic anhydrase |
| | | | | catalyzes reversible hydration of carbon dioxide. |
| | | | | Defects in this enzyme are associated with |
| | | | | osteopetrosis and renal tubular acidosis. |
| Platelet glycoprotein V | 27410 | 0.000493258 | IPI00027410 | GPV is part of the Ib-V-IX system of surface |
| precursor | | | | glycoproteins that constitute the receptor for von |
| | | | | Willebrand factor (VWF; MIM 193400) and |
| | | | | mediate the adhesion of platelets to injured |
| | | | | vascular surfaces in the arterial circulation, a |
| | | | | critical initiating event in hemostasis. |
| Protein DJ-1 | 298547 | 0.000536755 | IPI00298547 | human DJ1 could rescue Drosophila lacking |
| | | | | Dj1b, the fly homolog of DJ1, from oxidative |
| | | | | insult, and that a conserved cysteine (cys104, |
| | | | | which is analogous to human cys106) was critical |
| | | | | for antioxidant function in vivo. |
| Isoform 1 of Alpha-parvin | 18963 | 0.000777158 | IPI00018963 | family of proteins involved in linking integrins |
| | | | | and associated proteins with intracellular |
| | | | | pathways that regulate actin cytoskeletal |
| | | | | dynamics and cell survival. Both alpha-parvin |
| | | | | (PARVA) and beta-parvin (PARVB) localize to |
| | | | | focal adhesions and function in cell adhesion, |
| | | | | spreading, motility and survival through |
| | | | | interactions with partners, such as integrin-linked |
| | | | | kinase (ILK), paxillin, alpha-actinin and testicular |
| | | | | kinase 1. |
| TPM1 tropomyosin 1 | 216134 | 0.000935273 | IPI00216134 | contractile system of striated and smooth muscles |
| alpha chain isoform 7 | | | | and the cytoskeleton of non-muscle cells. |
| Calpain-1 catalytic | 11285 | 0.000997769 | IPI00011285 | The calpains, calcium-activated neutral proteases, |
| subunit | | | | are nonlysosomal, intracellular cysteine |
| | | | | proteases. The mammalian calpains include |
| | | | | ubiquitous, stomach-specific, and muscle-specific |
| | | | | proteins. |
| LOC390006 similar to | 374732 | 0.001005725 | IPI00374732 | This is listed as a pseudogene. |
| peptidylprolyl isomerase | | | | |
| A isoform 1 | | | | |
| MRPS31 28S ribosomal | 294242 | 0.002134587 | IPI00294242 | Mammalian mitochondrial ribosomal proteins are |
| protein S31, | | | | encoded by nuclear genes and help in protein |
| mitochondrial precursor | | | | synthesis within the mitochondrion. |
| | | | | Mitochondrial ribosomes (mitoribosomes) consist |
| | | | | of a small 28S subunit and a large 39S subunit. |
| | | | | They have an estimated 75% protein to rRNA |
| | | | | composition compared to prokaryotic ribosomes, |
| | | | | where this ratio is reversed. Another difference |
| | | | | between mammalian mitoribosomes and |
| | | | | prokaryotic ribosomes is that the latter contain a |
| | | | | 5S rRNA. |
| clathrin heavy chain 1 | 24067 | 0.002972246 | IPI00024067 | Clathrin is a major protein component of the |
| | | | | cytoplasmic face of intracellular organelles, |
| | | | | called coated vesicles and coated pits. These |
| | | | | specialized organelles are involved in the |
| | | | | intracellular trafficking of receptors and |
| | | | | endocytosis of a variety of macromolecules. |
| Peptidyl-prolyl cis-trans | 30144 | 0.003095306 | IPI00030144 | This is again a locus, but could be cyclophylin A |
| isomerase | | | | |
| bridging integrator 2 | 550792 | 0.003160967 | IPI0055092 | 564-amino acid protein has a BAR motif that is |
| | | | | 61% identical to that of BIN1 and slightly less |
| | | | | similar to that of AMPH (600418). BIN2 has |
| | | | | acidic and serine/proline-rich stretches but lacks a |
| | | | | C-terminal SH3 domain or a MYC-interacting |
| | | | | region. Northern blot analysis revealed |
| | | | | expression of a major 2.6-kb transcript that was |
| | | | | highest in spleen and peripheral blood leukocytes |
| | | | | and also high in thymus, colon, and placenta, |
| | | | | suggesting preferential expression in |
| | | | | hematopoietic tissues. Strong expression was |
| | | | | detected in lymphoid and granulocytic cell lines |
| | | | | but not other cell lines. |
| Coronin-1C | 8453 | 0.003354144 | IPI00008453 | Member of the WD repeat protein family. WD |
| | | | | repeats are minimally conserved regions of |
| | | | | approximately 40 amino acids typically bracketed |
| | | | | by gly-his and trp-asp (GH-WD), which may |
| | | | | facilitate formation of heterotrimeric or |
| | | | | multiprotein complexes. Members of this family |
| | | | | are involved in a variety of cellular processes, |
| | | | | including cell cycle progression, signal |
| | | | | transduction, apoptosis, and gene regulation. |
| Hypothetical protein | 3362 | 0.00392504 | IPI00003362 | When Chinese hamster K12 cells are starved of |
| | | | | glucose, the synthesis of several proteins, called |
| | | | | glucose-regulated proteins (GRPs), is markedly |
| | | | | increased. Hendershot et al. (1994) pointed out |
| | | | | that one of these, GRP78 (HSPA5), also referred |
| | | | | to as ‘immunoglobulin heavy chain-binding |
| | | | | protein’ (BiP), is a member of the heat-shock |
| | | | | protein-70 (HSP70) family and is involved in the |
| | | | | folding and assembly of proteins in the |
| | | | | endoplasmic reticulum (ER). ER stress? |
| ARPC1B Actin-related | 5160 | 0.004799351 | IPI00005160 | one of seven subunits of the human Arp2/3 |
| protein 2/3 complex | | | | protein complex. This subunit is a member of the |
| subunit 1B | | | | SOP2 family of proteins and is most similar to |
| | | | | the protein encoded by gene ARPC1A. The |
| | | | | similarity between these two proteins suggests |
| | | | | that they both may function as p41 subunit of the |
| | | | | human Arp2/3 complex that has been implicated |
| | | | | in the control of actin polymerization in cells. It is |
| | | | | possible that the p41 subunit is involved in |
| | | | | assembling and maintaining the structure of the |
| | | | | Arp2/3 complex. |
| Glutathione transferase | 19755 | 0.005077344 | IPI00019755 | theta class glutathione S-transferase-like |
| omega-1 | | | | (GSTTL) protein family. In mouse, the encoded |
| | | | | protein acts as a small stress response protein, |
| | | | | likely involved in cellular redox homeostasis. |
| Ras-related protein Rab- | 20436 | 0.005368216 | IPI00020436 | Almost nothing known. Divergent functions of |
| 11B | | | | neuronal Rab11b in Ca2+-regulated versus |
| | | | | constitutive exocytosis. |
| Rab GDP dissociation | 10154 | 0.006877841 | IPI00010154 | GDP dissociation inhibitors are proteins that |
| inhibitor alpha | | | | regulate the GDP-GTP exchange reaction of |
| | | | | members of the rab family, small GTP-binding |
| | | | | proteins of the ras superfamily, that are involved |
| | | | | in vesicular trafficking of molecules between |
| | | | | cellular organelles. GDIs slow the rate of |
| | | | | dissociation of GDP from rab proteins and release |
| | | | | GDP from membrane-bound rabs. GDI1 is |
| | | | | expressed primarily in neural and sensory tissues. |
| | | | | Mutations in GDI1 have been linked to X-linked |
| | | | | nonspecific mental retardation. |
| CDNA FLJ45525 fis, | 299571 | 0.007140832 | IPI00299571 | Involved in folding, ER stress response |
| clone BRTHA2026311, | | | | |
| highly similar to Protein | | | | |
| disulfide isomerase A6 | | | | |

TABLE 5
Proteins potentially associated with hemolysis (13):

| Protein | Accession | p val | Links | |
|---|---|---|---|---|
| ANK1 Isoform Er1 of | 216697 | 5.97539E−15 | IPI00216697 | associated with sudden cardiac death. RBC and |
| Ankyrin-1 | | | | epithelial protein. Heredary shpherocytosis. |
| SPTA1 Spectrin alpha | 220741 | 6.76561E−12 | IPI00220741 | actin crosslinking and molecular scaffold protein that |
| chain, erythrocyte | | | | links the plasma membrane to the actin cytoskeleton. |
| | | | | Mutations cause elliptocytosis type 2, |
| | | | | pyropoikilocytosis, and spherocytic hemolytic |
| | | | | anemia |
| EPB41 Isoform 1 of | 3921 | 6.87759E−06 | IPI00003921 | associated with elliptocytosis. Hereditary |
| Protein 4.1 | | | | elliptocytosis (HE) reflects a diminished elasticity of |
| | | | | the skeleton of RBC |
| Actin-like protein 2 | 5159 | 3.27694E−09 | IPI00005159 | arp2/3 complex, could be from hemolysis |
| Isoform 1 of Filamin-C | 178352 | 7.95487E−07 | IPI00178352 | Gamma filamin crosslinks actin filaments into |
| | | | | orthogonal networks in cortical cytoplasm and |
| | | | | participate in the anchoring of membrane proteins |
| | | | | for the actin cytoskeleton. |
| Filamin A, alpha | 302592 | 1.89688E−05 | IPI00302592 | Filamin A is a 280-kD protein that crosslinks actin |
| | | | | filaments into orthogonal networks in cortical |
| | | | | cytoplasm and participates in the anchoring of |
| | | | | membrane proteins for the actin cytoskeleton. |
| | | | | Remodeling of the cytoskeleton is central to the |
| | | | | modulation of cell shape and migration. Filamin A, |
| | | | | encoded by the FLNA gene, is a widely expressed |
| | | | | protein that regulates reorganization of the actin |
| | | | | cytoskeleton by interacting with integrins, |
| | | | | transmembrane receptor complexes, and second |
| | | | | messengers. |
| F-actin capping protein | 5969 | 0.000842366 | IPI00005969 | Member of the F-actin capping protein alpha subunit |
| alpha-1 subunit | | | | family. This gene encodes the alpha subunit of the |
| | | | | barbed-end actin binding protein. |
| PDZ and LIM domain | 10414 | 0.001546966 | IPI00010414 | PDLIM1 interacts with the C-terminal EF-hand |
| protein 1 | | | | region of alpha-actinin-2 |
| Fructose-bisphosphate | 465439 | 0.00317286 | IPI00465439 | Hemolytic anemia and severe rhabdomyolysis |
| aldolase A | | | | caused by compound heterozygous mutations of the |
| | | | | gene for erythrocyte/muscle isozyme of aldolase, |
| | | | | ALDOA. glycolytic enzyme that catalyzes the |
| | | | | reversible conversion of fructose-1,6-bisphosphate to |
| | | | | glyceraldehyde 3-phosphate and dihydroxyacetone |
| | | | | phosphate |
| Band 3 anion transport | 22361 | 2.25331E−06 | IPI00022361 | Band 3. Another marker of hemolysis. CD233 is |
| protein | | | | expressed in the erythrocyte plasma membrane |
| | | | | where it functions as a chloride/bicarbonate |
| | | | | exchanger involved in carbon dioxide transport from |
| | | | | tissues to lungs. |
| EPB42 Isoform Long of | 28120 | 0.004833753 | IPI00028120 | Erythrocyte membrane protein band 4.2 is an ATP- |
| Erythrocyte membrane | | | | binding protein which may regulate the association |
| protein band 4.2 | | | | of protein 3 with ankyrin. |
| Isoform 1 of F-actin | 26185 | 0.000120575 | IPI00026185 | The protein regulates growth of the actin filament by |
| capping protein subunit | | | | capping the barbed end of growing actin filaments |
| beta | | | | |
| PKLR Isoform R-type | 27165 | 0.000136699 | IPI00027165 | Pyruvate kinase that catalyzes the production of |
| of Pyruvate kinase | | | | phosphoenolpyruvate from pyruvate and ATP. |
| isozymes R/L | | | | Defects in this enzyme, due to gene mutations or |
| | | | | genetic variations, are the common cause of chronic |
| | | | | hereditary nonspherocytic hemolytic anemia |
| | | | | (CNSHA or HNSHA). |

TABLE 6
Proteins Underexpressed in AAA (29)

| Protein | Accession | p val | Links | assoc AAA | |
|---|---|---|---|---|---|
| von | 23014 | 2.88E−07 | IPI00023014 | yes, but Ihara A, | The glycoprotein encoded by this gene |
| Willebrand | | | | Matsumoto K, | functions as both an antihemophilic factor |
| factor | | | | Kawamoto T, | carrier and a platelet-vessel wall mediator in the |
| precursor | | | | Shouno S, | blood coagulation system. It is crucial to the |
| | | | | Kawamoto J, | hemostasis process. Mutations in this gene or |
| | | | | Katayama A, | deficiencies in this protein result in von |
| | | | | Yoshitatsu M, | Willebrand's disease. The reduction probably |
| | | | | Izutani H. | means that fewer MPs are from platelets and |
| | | | | report as | ECs. |
| | | | | overexpressed | |
| | | | | in solution | |
| Galectin-3- | 23673 | 3.04E−07 | IPI00023673 | This is a | Also known as Mac-2, overexpressed in |
| binding | | | | biomarker of | patients with CAD. Since most controls have |
| protein | | | | atherosclerosis: | CAD, this makes sense. |
| precursor | | | | maybe co- | |
| | | | | morbidity | |
| apolipoprotein | 299435 | 5.57E−07 | IPI00299435 | no | minor lipoprotein in plasma, only 8 papers in |
| F precursor | | | | | PubMed. Molecular cloning and expression of |
| | | | | | lipid transfer inhibitor protein reveals its |
| | | | | | identity with apolipoprotein F. A related protein |
| | | | | | is associated with phospholipid transfer in mice |
| | | | | | (Paigen). |
| Clusterin | 291262 | 3.34E−06 | IPI00291262 | no | not yet clear. it is known to be expressed in a |
| precursor | | | | | variety of tissues and it seems to be able to bind |
| | | | | | to cells, membranes and hydrophobic proteins. |
| | | | | | it has been associated with programmed cell |
| | | | | | death (apoptosis). Maybe also in DNA repair. |
| Hypothetical | 399007 | 5.53E−06 | IPI00399007 | no | Essentially a duplication of the IgG2 gene. |
| protein | | | | | Homozygosity for the G2M*n allele is strongly |
| DKFZp686I04196 | | | | | associated with absence of severe infections in |
| (Fragment) | | | | | Swedish patients with hereditary complement |
| | | | | | 2-deficiency and suggests a major protective |
| | | | | | role |
| ALB protein | 216773 | 6.21E−06 | IPI00216773 | no | This is just serum albumin. Unclear why |
| | | | | | underexpressed in AAA |
| IGKV1-5 | 419424 | 1.48E−05 | IPI00419424 | no | Only 2 papers in Puib Med. Associate with |
| protein | | | | | leukemias. |
| IGLC1 protein | 154742 | 2.68E−05 | IPI00154742 | no | IGLC1 not in PubMed |
| FLJ00385 | 168728 | 4.76E−05 | IPI00168728 | no | This is IgM heavy chain. Maybe |
| protein | | | | | immunocomplexes? |
| (Fragment) | | | | | |
| Complement | 32258 | 9.16E−05 | IPI00032258 | no | This gene encodes the acidic form of |
| C4-A | | | | | complement factor 4, part of the classical |
| precursor | | | | | activation pathway. The protein is expressed as |
| | | | | | a single chain precursor which is proteolytically |
| | | | | | cleaved into a trimer of alpha, beta, and gamma |
| | | | | | chains prior to secretion. The trimer provides a |
| | | | | | surface for interaction between the antigen- |
| | | | | | antibody complex and other complement |
| | | | | | components. The alpha chain may be cleaved to |
| | | | | | release C4 anaphylatoxin, a mediator of local |
| | | | | | inflammation. Deficiency of this protein is |
| | | | | | associated with systemic lupus erythematosus |
| | | | | | and type I diabetes mellitus. |
| IGHA1 | 61977 | 0.000121 | IPI00061977 | no | IgA heavy chain |
| protein | | | | | |
| Ig mu heavy | 385264 | 0.000165 | IPI00385264 | no | Ig alpha-1 chain C region |
| chain disease | | | | | |
| protein | | | | | |
| IGHM protein | 477090 | 0.000594 | IPI00477090 | no | IgM heavy chain |
| 19 kDa protein | 472787 | 0.000695 | IPI00472787 | no | not in database, must blast |
| Factor VII | 382606 | 0.000757 | IPI00382606 | yes, but the | coagulation factor VII |
| active site | | | | papers are on | |
| mutant | | | | treatment | |
| immunoconjugate | | | | after | |
| | | | | hemorrhage | |
| Vitronectin | 298971 | 0.000807 | IPI00298971 | yes: | member of the pexin family. It is found in |
| precursor | | | | Immunoreactivity | serum and tissues and promotes cell adhesion |
| | | | | of | and spreading, inhibits the membrane-damaging |
| | | | | adventitial | effect of the terminal cytolytic complement |
| | | | | matrix fibrils | pathway, and binds to several serpin serine |
| | | | | of normal and | protease inhibitors. It is a secreted protein and |
| | | | | aneurysmal | exists in either a single chain form or a clipped, |
| | | | | abdominal | two chain form held together by a disulfide |
| | | | | aorta with | bond. |
| | | | | antibodies | |
| | | | | against | |
| | | | | vitronectin | |
| | | | | and | |
| | | | | fibrinogen. | |
| ALB protein | 22434 | 0.001287 | IPI00022434 | no | serum albumin |
| PREDICTED: | 297263 | 0.001822 | IPI00297263 | no | human homolog of zebrafish HEG1. Only 2 |
| HEG homolog 1 | | | | | papers, both irrelevant |
| Transferrin | 22462 | 0.002686 | IPI00022462 | Yes. Elevated | we have seen this protein previously. |
| receptor | | | | transferrin | Underexpression may reflect “dilution” of |
| protein 1 | | | | concentration | “normal” MPs. |
| | | | | in cerebral | |
| | | | | spinal fluid | |
| | | | | after | |
| | | | | subarachnoid | |
| | | | | hemorrhage. | |
| Apolipoprotein | 21841 | 0.003472 | IPI00021841 | no | apoA1 |
| A-I | | | | | |
| precursor | | | | | |
| C4b-binding | 21727 | 0.00389 | IPI00021727 | no | interesting: May form a complex with C4. |
| protein alpha | | | | | Tandemly arrayed short consensus repeats of |
| chain | | | | | approximately 60 amino acids. Along with a |
| precursor | | | | | single, unique beta-chain, seven identical alpha- |
| | | | | | chains encoded by this gene assemble into the |
| | | | | | predominant isoform of C4b-binding protein, a |
| | | | | | multimeric protein that controls activation of |
| | | | | | the complement cascade through the classical |
| | | | | | pathway. |
| Apolipoprotein | 21842 | 0.003992 | IPI00021842 | false positive | apoE. Could be from macrophages or liver |
| E precursor | | | | come up | |
| | | | | because of | |
| | | | | apoE KO | |
| | | | | mouse. | |
| Ig kappa chain | 30205 | 0.004157 | IPI00030205 | no | Two papers show different distribution in |
| V-III region | | | | | leukemias. |
| HAH | | | | | |
| precursor | | | | | |
| Carbonic | 215983 | 0.005354 | IPI00215983 | no (4 false | zinc metalloenzymes that catalyze the |
| anhydrase 1 | | | | positives in | reversible hydration of carbon dioxide. They |
| | | | | PubMed) | participate in a variety of biological processes, |
| | | | | | including respiration, calcification, acid-base |
| | | | | | balance, bone resorption, and the formation of |
| | | | | | aqueous humor, cerebrospinal fluid, saliva, and |
| | | | | | gastric acid. |
| Syntenin-1 | 299086 | 0.007591 | IPI00299086 | no | Links syndecan-mediated signaling to the |
| | | | | | cytoskeleton. The syntenin protein contains |
| | | | | | tandemly repeated PDZ domains that bind the |
| | | | | | cytoplasmic, C-terminal domains of a variety of |
| | | | | | transmembrane proteins. This protein may also |
| | | | | | affect cytoskeletal-membrane organization, cell |
| | | | | | adhesion, protein trafficking, and the activation |
| | | | | | of transcription factors. The protein is primarily |
| | | | | | localized to membrane-associated adherens |
| | | | | | junctions and focal adhesions but is also found |
| | | | | | at the endoplasmic reticulum and nucleus. |
| PRO2275 | 305457 | 0.008362 | IPI00305457 | Yes. Ruptured | Alpha-1-antitrypsin is a protease inhibitor, |
| | | | | mesenteric | deficiency of which is associated with |
| | | | | artery | emphysema and liver disease. |
| | | | | aneurysm in a | |
| | | | | patient with | |
| | | | | alpha 1- | |
| | | | | antitrypsin | |
| | | | | deficiency: | |
| | | | | etiologic | |
| | | | | implications. | |
| Apolipoprotein | 304273 | 0.008786 | IPI00304273 | no | Synthesis in the intestine, in association with |
| A-IV | | | | | chylomicron particles. Although its precise |
| precursor | | | | | function is not known, apo A-IV is a potent |
| | | | | | activator of lecithin-cholesterol acyltransferase |
| | | | | | in vitro. |
| Isoform 2 of | 241562 | 0.008941 | IPI00241562 | no | Large secreted extracellular matrix protein |
| Reelin | | | | | thought to control cell-cell interactions critical |
| precursor | | | | | for cell positioning and neuronal migration |
| | | | | | during brain development. This protein may be |
| | | | | | involved in schizophrenia, autism, bipolar |
| | | | | | disorder, major depression and in migration |
| | | | | | defects associated with temporal lobe epilepsy. |

TABLE 7
Patient Demographics: Cases

| Age | Sex | Race | AAA size | HTN | smoking | DM | CA | CAD | Lipids | Other |
|---|---|---|---|---|---|---|---|---|---|---|
| 72 | M | C | 5.5 | X | Current, 60 | | | | X | Eczema |
| | | | | | | | | | | arthritis, |
| | | | | | | | | | | cerebral |
| | | | | | | | | | | aneurysm |
| 67 | M | C | 7.5 | X | 100 | X + O | | X | X | COPD |
| | | | | | | | | | | CHF |
| 61 | M | C | 4.4 | X | Current | | | X | X | COPD |
| | | | | | | | | | | TIA |
| 79 | F | C, H | 5.8 | X | 60 | | br | | | COPD |
| 68 | M | C | 4.3 + iliac | X | 150 | X + O | | | X | COPD, |
| | | | | | | | | | | CRI |
| | | | | | | | | | | CHF |

TABLE 8
Controls:

| Age | Sex | Race | Aneurysm | HTN | Smoking | DM | CA | CAD | Lipids | Other |
|---|---|---|---|---|---|---|---|---|---|---|
| 60 | M | C | | X | Current | | pros | X | X | GERD |
| 73 | F | C | | X | Quit74 | X + O | | X | X | PE |
| | | | | | | | | | | Afib |
| | | | | | | | | | | CHF |
| 75 | F | C | | X | Not current | | | X | X | PUD |
| | | | | | | | | | | Depression |
| 58 | M | C | | X | Quit | | | X | X | PCK |
| 69 | M | C | | X | Quit | X + I | | X | | COPD |
| | | | | | | | | | | CVA |
| 73 | M | C | | | Quit 83 | | | X | X | |

Other methods which were used but not described herein are well known and within the competence of one of ordinary skill in the art of cell biology, molecular biology, and clinical medicine. The invention should not be construed to be limited solely to the assays and methods described herein, but should be construed to include other methods and assays as well. One of skill in the art will know that other assays and methods are available to perform the procedures described herein.

Headings are included herein for reference and to aid in locating certain sections. These headings are not intended to limit the scope of the concepts described therein under, and these concepts may have applicability in other sections throughout the entire specification. The disclosures of each and every patent, patent application, and publication cited herein are hereby incorporated herein by reference in their entirety. While this invention has been disclosed with reference to specific embodiments, it is apparent that other embodiments and variations of this invention may be devised by the previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

### Bibliography

1. Diehm et al., 2007, Prog. Cardiovasc. Dis. 50:209-217.
2. Sandford et al., 2007, Eur. J. Vasc. Endovasc. Surg. 33:381-390.
3. Ye, 2006, Cardiovasc. Res. 69:636-645.
4. Pearce and Shively, 2006, Ann. N. Y. Acad. Sci. 1085:117-32.:117-132.
5. Eliason et al., 2005, Circulation 112:232-240.
6. Daugherty et al., 2000, J. Clin. Invest. 105:1605-1612.
7. Cassis et al., 200, Arterioscler. Thromb. Vasc. Biol. 27:380-386.
8. Chaer et al., 2006, Vascular. 14:343-352.
9. Smalley et al., 2007, Thromb. Haemost. 97:67-80.
10. Smalley and Ley, 2007, Clinical Laboratory in press [Inventors—Is this still in press?].
11. Garcia et al., 2005, J. Proteome. Res. 4:1516-1521.
12. Coon et al., 2005, Biotechniques 38:519, 521, 523.
13. Anderson and Anderson, 2002, Mol. Cell Proteomics. 1:845-867.
14. Bjorhall et al., 2005, Proteomics. 5:307-317.
15. Liu et al., 2004, Anal. Chem. 76:4193-4201.
16. Hynes, 1992, Cell 69:11-25.
17. Berton et al., 1996, International Journal of Clinical & Laboratory Research 26:160-177.
18. Hynes, 2002, Cell 110:673-687.
19. Legate et al., 2006, Nat. Rev. Mol. Cell Biol. 7:20-31.
20. Hannigan et al., 1996, Nature 379:91-96.
21. Brown et al., 1999, J. Biol Chem. 274:27083-27091.
22. Tadokoro et al., 2003, Science 302:103-106.
23. Rose et al., 2007, Immunol. Rev. 218:126-34.:126-134.
24. Critchley, 2005, Biochem. Soc. Trans. 33:1308-1312.
25. Franco et al., 2004, Nat. Cell Biol 6:977-983.
26. Hayward, 1997, Clin. Invest Med. 20:176-187.
27. Sperandio, 2003, J. Exp. Med. 197:1355-1363.
28. Schmidtke and Diamond, 2000, J. Cell Biol. 149:719-730.
29. Tabas, 2005, Thromb. Vasc. Biol. 25:2255-2264.

What is claimed is:

1. A method of diagnosing an abdominal aortic aneurysm, said method comprising obtaining a biological sample from a test subject, comparing the level of at least one protein biomarker associated with abdominal aortic aneurysm in said test subject with the level of said protein biomarker from an otherwise identical sample obtained from an unaffected subject or with a standard sample comprising a known amount of said protein biomarker, wherein a higher or lower level of said protein biomarker in said sample from a test subject compared with the level of said protein biomarker in said sample from an unaffected subject or from said standard, is an indication that said test subject has an abdominal aortic aneurysm, thereby diagnosing an abdominal aortic aneurysm.

2. The method of claim 1, wherein said sample is selected from the group consisting of tissue samples, biopsies, blood, plasma, saliva, feces, cerebrospinal fluid, semen, tears, and urine.

3. The method of claim 2, wherein said sample is plasma.

4. The method of claim 3, wherein said plasma is processed to obtain plasma-derived microparticles.

5. The method of claim 4, wherein said protein levels are measured using said microparticles.

6. The method of claim 1, wherein at least one of said protein biomarkers is one which is found at higher levels in a subject with an abdominal aortic aneurysm compared with the level of said protein biomarker in an unaffected subject.

7. The method of claim 6, wherein said protein biomarker found at higher levels in a subject with an abdominal aortic aneurysm is selected from the group consisting of 14-3-3 protein epsilon, 14-3-3 protein eta, 14-3-3 protein gamma, 14-3-3 protein zeta/delta, 271 kDa protein, Actin-like protein 3, ACTN4 Alpha-actinin-4, Adenylyl cyclase-associated protein 1, Alpha-actinin-1, ARPC1B-Actin-related protein ⅔ complex subunit 1B, ARPC2 PNAS-139, ARPC4 Actin-related protein ⅔ complex subunit 4, Beta-parvin, Bridging integrator 2, Calpain-1 catalytic subunit, CANX Calnexin precursor, CAPZA2 F-actin capping protein subunit alpha-2, Carbonic anhydrase 2, CDNA FLJ45525 fis-clone BRTHA2026311-highly similar to Protein disulfide isomerase A6, Chloride intracellular channel protein 1, clathrin heavy chain 1, Coagulation factor XIII A chain precursor, Cofilin-1, Coronin-1C, COTL1 Coactosin-like protein, EDARADD ENO1P protein, EHD1 EH domain-containing protein 1, EHD3 EH domain-containing protein 3, Endoplasmin precursor, enolase 1, FHL 1 Four and a half LIM domains 1 variant, FLNB Isoform 1 of Filamin-B, Glutathione S-transferase P, Glutathione transferase omega-1, Glyceraldehyde-3-phosphate dehydrogenase, GPX1 glutathione peroxidase 1 isoform 1, Heat shock protein 86 (Fragment), HSPA4 Heat shock 70 kDa protein 4, HSPC159 Galectin-related protein, Hypothetical protein, Hypothetical protein DKFZp761 K0511, Hypothetical protein FLJ25678, IDH2 Isocitrate dehydrogenase [NADP]-mitochondrial precursor, Integrin-linked protein kinase 1, Isoform 1 of Alpha-parvin, Isoform 1 of Gelsolin precursor, Isoform 1 of Heat shock cognate 71 kDa protein, Isoform 1 of Vinculin, Isoform 2 of Unc-112-related protein 2, Isoform Beta-3B of Integrin beta-3 precursor, Isoform M1 of Pyruvate kinase isozymes M1/M2, Isoform SERCA3B of Sarcoplasmic/endoplasmic reticulum calcium ATPase 3, KCNK15 Potassium channel subfamily K member 15, Lactate dehydrogenase A, Leukocyte elastase inhibitor, L-lactate dehydrogenase B chain, LOC390006 similar to peptidylprolyl isomerase A isoform 1, LTBP1 Latent-transforming growth factor beta-binding protein-isoform IL precursor, LYAR Cell growth-regulating nucleolar protein, MAPRE2 Isoform 1 of Microtubule-associated protein RP/EB family member 2, MRPS30 28S ribosomal protein S31-mitochondrial precursor, Multimerin-1 precursor, Myosin regulatory light chain, Myosin regulatory light polypeptide 9 isoform b, Myosin-9, NME1 Nucleoside diphosphate kinase A, Peptidyl-prolyl cis-trans isomerase, Peptidylprolyl isomerase B precursor, Phosphoglycerate kinase 1, PINCH protein, Platelet glycoprotein V precursor, Pleckstrin, Protein disulfide-isomerase precursor, Protein DJ-1, PTGS1 Cyclooxygenase 1b3, Rab GDP dissociation inhibitor alpha, RAB6B Ras-related protein Rab-6B, RAC2 Ras-related C3 botulinum toxin substrate 2 precursor, Ras suppressor protein 1, Ras-related protein Rab-11B, Ras-related protein Rab-27B, RcTPI1 (Fragment), Rho GDP-dissociation inhibitor 2, RTN4 Isoform 1 of Reticulon-4, S100A4 Protein S100-A4, SELP P-selectin precursor, SEPT11 Septin-11, Serum deprivation-response protein, SLC2A3 Solute carrier family 2-facilitated glucose transporter member 3, SOD1 16 kDa protein, SPARC SPARC precursor, SPTBN5 Spectrin beta chain-brain 4, STXBP2 Syntaxin-binding protein 2, Superoxide dismutase [Mn]-mitochondrial precursor, TBXAS1 thromboxane A synthase 1 (platelet, cytochrome P450, family 5, subfamily A) isoform TXS-II, Thrombospondin-1 precursor, Thrombospondin-2 precursor, TPI1 Isoform 2 of Triosephosphate isomerase, TPM1 tropomyosin 1 alpha chain isoform 7, Transgelin-2, Transitional endoplasmic reticulum ATPase, Tropomyosin 4, Tubulin alpha-1 chain, Tubulin beta-1 chain, Tubulin beta-2C chain, and VASP Vasodilator-stimulated phosphoprotein.

8. The method of claim 7, wherein at least one of the protein biomarkers is FHL1 Four and a half LIM domains 1 variant.

9. The method of claim 7, wherein at least one of the protein biomarkers is COTL1 Coactosin-like protein.

10. The method of claim 6, wherein said at least one protein biomarker which is found at higher levels in a subject with an abdominal aortic aneurysm is selected from the group consisting of heat shock proteins, proteins related to extracellular matrix, proteins related to inflammation, proteins involved in metabolic processes, cytoskeletal proteins, proteins associated with endoplasmic reticulum, ion pumps and channels, proteins involved in angiogenesis, small GTPases, mast cell-associated proteins, platelet-associated proteins, surface receptor or associated proteins, proteins involved in calcium handling, oxidative stress proteins, mitochondrial proteins, signaling proteins, nuclear proteins, transcription factors, and proteins potentially associated with hemolysis.

11. The method of claim 10, wherein said proteins potentially associated with hemolysis are selected from the group consisting of ANK1 Isoform Er1 of Ankyrin-1, SPTA1 Spectrin alpha chain-erythrocyte, EPB41 Isoform 1 of Protein 4.1, Actin-like protein 2, Isoform 1 of Filamin-C, Filamin A alpha, F-actin capping protein alpha-1 subunit, PDZ and LIM domain protein 1, Fructose-bisphosphate aldolase A, Band 3 anion transport protein, EPB42 Isoform Long of Erythrocyte membrane protein band 4.2, Isoform 1 of F-actin capping protein subunit beta, and PKLR Isoform R-type of Pyruvate kinase isozymes R/L.

12. The method of claim 1, wherein at least one of said protein biomarkers is one which is found at lower levels in a subject with an abdominal aortic aneurysm compared with the level of said protein biomarker in an unaffected subject.

13. The method of claim 11, wherein at least one of said protein biomarkers which is found at lower levels is selected from the group consisting of 19 kDa protein, ALB protein (22434), ALB Protein (216773), Apolipoprotein A-I precursor, Apolipoprotein A-IV precursor, Apolipoprotein E precursor, Apolipoprotein F precursor, C4b-binding protein alpha chain precursor, Carbonic anhydrase 1, Clusterin precursor, Complement C4-A precursor, Factor VII active site mutant immunoconjugate, FLJ00385 protein (Fragment), Galectin-3-binding protein precursor, Hypothetical protein DKFZp686I04196 (Fragment), Ig kappa chain V-III region HAH precursor, Ig mu heavy chain disease protein, IGHA1 protein, IGHM protein, IGKV1-5 protein, IGLC1 protein, Isoform 2 of Reelin precursor, PREDICTED: HEG homolog 1, PRO2275, Syntenin-1, Transferrin receptor protein 1, Vitronectin precursor, and von Willebrand factor precursor.

14. The method of claim 1, wherein the levels of at least two protein biomarkers are compared.

15. The method of claim 14, wherein at least one of said protein biomarkers is one which is found at higher levels in a subject with an abdominal aortic aneurysm compared with the level of said protein biomarker in an unaffected subject.

16. The method of claim 15, wherein the at least one of said protein biomarkers found at higher levels in a subject with an abdominal aortic aneurysm is selected from the group consisting of 14-3-3 protein epsilon, 14-3-3 protein eta, 14-3-3 protein gamma, 14-3-3 protein zeta/delta, 271 kDa protein, Actin-like protein 3, ACTN4 Alpha-actinin-4, Adenylyl cyclase-associated protein 1, Alpha-actinin-1, ARPC1B-Actin-related protein ⅔ complex subunit 1B, ARPC2 PNAS-139, ARPC4 Actin-related protein ⅔ complex subunit 4, Beta-parvin, Bridging integrator 2, Calpain-1 catalytic subunit, CANX Calnexin precursor, CAPZA2 F-actin capping protein subunit alpha-2, Carbonic anhydrase 2, CDNA FLJ45525 fis-clone BRTHA2026311-highly similar to Protein disulfide isomerase A6, Chloride intracellular channel protein 1, clathrin heavy chain 1, Coagulation factor XIII A chain precursor, Cofilin-1, Coronin-1C, COTL1 Coactosin-like protein, EDARADD ENO1P protein, EHD1 EH domain-containing protein 1, EHD3 EH domain-containing protein 3, Endoplasmin precursor, enolase 1, FHL1 Four and a half LIM domains 1 variant, FLNB Isoform 1 of Filamin-B, Glutathione S-transferase P, Glutathione transferase omega-1, Glyceraldehyde-3-phosphate dehydrogenase, GPX1 glutathione peroxidase 1 isoform 1, Heat shock protein 86 (Fragment), HSPA4 Heat shock 70 kDa protein 4, HSPC159 Galectin-related protein, Hypothetical protein, Hypothetical protein DKFZp761K0511, Hypothetical protein FLJ25678, IDH2 Isocitrate dehydrogenase [NADP]-mitochondrial precursor, Integrin-linked protein kinase 1, Isoform 1 of Alpha-parvin, Isoform 1 of Gelsolin precursor, Isoform 1 of Heat shock cognate 71 kDa protein, Isoform 1 of Vinculin, Isoform 2 of Unc-112-related protein 2, Isoform Beta-3B of Integrin beta-3 precursor, Isoform M1 of Pyruvate kinase isozymes M1/M2, Isoform SERCA3B of Sarcoplasmic/endoplasmic reticulum calcium ATPase 3, KCNK15 Potassium channel subfamily K member 15, Lactate dehydrogenase A, Leukocyte elastase inhibitor, L-lactate dehydrogenase B chain, LOC390006 similar to peptidylprolyl isomerase A isoform 1, LTBP1 Latent-transforming growth factor beta-binding protein-isoform IL precursor, LYAR Cell growth-regulating nucleolar protein, MAPRE2 Isoform 1 of Microtubule-associated protein RP/EB family member 2, MRPS30 28S ribosomal protein S31-mitochondrial precursor, Multimerin-1 precursor, Myosin regulatory light chain, Myosin regulatory light polypeptide 9 isoform b, Myosin-9, NME1 Nucleoside diphosphate kinase A, Peptidyl-prolyl cis-trans isomerase, Peptidylprolyl isomerase B precursor, Phosphoglycerate kinase 1, PINCH protein, Platelet glycoprotein V precursor, Pleckstrin, Protein disulfide-isomerase precursor, Protein DJ-1, PTGS1 Cyclooxygenase 1b3, Rab GDP dissociation inhibitor alpha, RAB6B Ras-related protein Rab-6B, RAC2 Ras-related C3 botulinum toxin substrate 2 precursor, Ras suppressor protein 1, Ras-related protein Rab-11B, Ras-related protein Rab-27B, RcTPI1 (Fragment), Rho GDP-dissociation inhibitor 2, RTN4 Isoform 1 of Reticulon-4, S100A4 Protein S100-A4, SELP P-selectin precursor, SEPT11 Septin-11, Serum deprivation-response protein, SLC2A3 Solute carrier family 2-facilitated glucose transporter member 3, SOD1 16 kDa protein, SPARC SPARC precursor, SPTBN5 Spectrin beta chain-brain 4, STXBP2 Syntaxin-binding protein 2, Superoxide dismutase [Mn]-mitochondrial precursor, TBXAS1 thromboxane A synthase 1 (platelet, cytochrome P450, family 5, subfamily A) isoform TXS-II, Thrombospondin-1 precursor, Thrombospondin-2 precursor, TPI1 Isoform 2 of Triosephosphate isomerase, TPM1 tropomyosin 1 alpha chain isoform 7, Transgelin-2, Transitional endoplasmic reticulum ATPase, Tropomyosin 4, Tubulin alpha-1 chain, Tubulin beta-1 chain, Tubulin beta-2C chain, and VASP Vasodilator-stimulated phosphoprotein.

17. The method of claim 16, wherein at least one of the protein biomarkers is FHL1 Four and a half LIM domains 1 variant.

18. The method of claim 16, wherein at least one of the protein biomarkers is COTL1 Coactosin-like protein.

19. The method of claim 15, wherein said at least one protein biomarker which is found at higher levels in a subject with an abdominal aortic aneurysm is selected from the group consisting of heat shock proteins, proteins related to extracellular matrix, proteins related to inflammation, proteins involved in metabolic processes, cytoskeletal proteins, proteins associated with endoplasmic reticulum, ion pumps and channels, proteins involved in angiogenesis, small GTPases, mast cell-associated proteins, platelet-associated proteins, surface receptor or associated proteins, proteins involved in calcium handling, oxidative stress proteins, mitochondrial proteins, signaling proteins, nuclear proteins, transcription factors, and proteins potentially associated with hemolysis.

20. The method of claim 19, wherein said proteins potentially associated with hemolysis are selected from the group consisting of ANK1 Isoform Er1 of Ankyrin-1, SPTA1 Spectrin alpha chain-erythrocyte, EPB41 Isoform 1 of Protein 4.1, Actin-like protein 2, Isoform 1 of Filamin-C, Filamin A alpha, F-actin capping protein alpha-1 subunit, PDZ and LIM domain protein 1, Fructose-bisphosphate aldolase A, Band 3 anion transport protein, EPB42 Isoform Long of Erythrocyte membrane protein band 4.2, Isoform 1 of F-actin capping protein subunit beta, and PKLR Isoform R-type of Pyruvate kinase isozymes R/L.

21. The method of claim 14, wherein at least one of the protein biomarkers is one which is found at lower levels in a subject with an abdominal aortic aneurysm compared with the level of said protein biomarker in an unaffected subject.

22. The method of claim 21, wherein at least one of said protein biomarkers which is found at lower levels is selected from the group of protein biomarkers consisting of 19 kDa protein, ALB protein (22434), ALB Protein (216773), Apolipoprotein A-I precursor, Apolipoprotein A-IV precursor, Apolipoprotein E precursor, Apolipoprotein F precursor, C4b-binding protein alpha chain precursor, Carbonic anhydrase 1, Clusterin precursor, Complement C4-A precursor, Factor VII active site mutant immunoconjugate, FLJ00385 protein (Fragment), Galectin-3-binding protein precursor, Hypothetical protein DKFZp686I04196 (Fragment), Ig kappa chain V-III region HAH precursor, Ig mu heavy chain disease protein, IGHA1 protein, IGHM protein, IGKV1-5 protein, IGLC1 protein, Isoform 2 of Reelin precursor, PREDICTED: HEG homolog 1, PRO2275, Syntenin-1, Transferrin receptor protein 1, Vitronectin precursor, and von Willebrand factor precursor.

23. The method of claim 14, wherein at least one of said at least two protein biomarkers is one which is found at higher levels in a subject with an abdominal aortic aneurysm compared with the level of said protein biomarker in an unaffected subject, and at least one of said at least two protein biomarkers is one which is found at lower levels in a subject with an abdominal aortic aneurysm compared with the level of said protein biomarker in an unaffected subject.

24. The method of claim 1, wherein said test subject is a human.

25. The method of claim 1, wherein said test subject is at risk for developing an abdominal aortic aneurysm.

26. The method of claim 25, wherein the test subject has at least one risk factor for developing an abdominal aortic aneurysm, said at least one risk factor selected from the group consisting of high blood pressure, smoking, high cholesterol, emphysema, genetic factors, age, and male gender.

27. The method of claim 1, wherein said test subject is asymptomatic for an abdominal aortic aneurysm.

28. The method of claim 1, wherein said test subject is symptomatic for an abdominal aortic aneurysm.

29. The method of claim 1, wherein said protein biomarker levels are measured using a technique selected from the group consisting of flow cytometry, western blots, immunoblots, ELISA, MS/MS spectroscopy, and biological activity assays.

30. A method of monitoring the progression of an abdominal aortic aneurysm in a subject previously diagnosed with an abdominal aortic aneurysm, said method comprising:
a) measuring the level of at least one protein biomarker associated with an abdominal aortic aneurysm in a first biological sample obtained from said subject to determine an initial level of said protein biomarker;
b) measuring the level of said protein biomarker in a second otherwise identical biological sample obtained from said subject at a later point in time,
d) comparing the level of said protein biomarker in said first biological sample with the level of said protein biomarker in said second otherwise identical biological sample obtained from said subject; and
e) correlating any change in the level of said protein biomarker in said second otherwise identical biological sample with the progression of the abdominal aortic aneurysm,
thereby monitoring the progression of an abdominal aortic aneurysm in a subject.

31. The method of claim 30, wherein said protein biomarker levels are measured more than once following the measurement of the initial levels of said protein biomarker.

32. The method of claim 30, wherein at least one of said protein biomarkers is one which is found at higher levels in a subject with an abdominal aortic aneurysm compared with the level of said protein biomarker in an unaffected subject.

33. The method of claim 30, wherein at least one of said protein biomarkers is one which is found at lower levels in a subject with an abdominal aortic aneurysm compared with the level of said protein biomarker in an unaffected subject.

34. The method of claim 30, wherein at least two protein biomarkers are measured.

35. An abdominal aortic aneurysm biomarker selected from the group consisting of 14-3-3 protein epsilon, 14-3-3 protein eta, 14-3-3 protein gamma, 14-3-3 protein zeta/delta, 271 kDa protein, Actin-like protein 3, ACTN4 Alpha-actinin-4, Adenylyl cyclase-associated protein 1, Alpha-actinin-1, ARPC1B-Actin-related protein ⅔ complex subunit 1B, ARPC2 PNAS-139, ARPC4 Actin-related protein ⅔ complex subunit 4, Beta-parvin, Bridging integrator 2, Calpain-1 catalytic subunit, CANX Calnexin precursor, CAPZA2 F-actin capping protein subunit alpha-2, Carbonic anhydrase 2, CDNA FLJ45525 fis-clone BRTHA2026311-highly similar to Protein disulfide isomerase A6, Chloride intracellular channel protein 1, clathrin heavy chain 1, Coagulation factor XIII A chain precursor, Cofilin-1, Coronin-1C, COTL1 Coactosin-like protein, EDARADD ENO1P protein, EHD1 EH domain-containing protein 1, EHD3 EH domain-containing protein 3, Endoplasmin precursor, enolase 1, FHL1 Four and a half LIM domains 1 variant, FLNB Isoform 1 of Filamin-B, Glutathione S-transferase P, Glutathione transferase omega-1, Glyceraldehyde-3-phosphate dehydrogenase, GPX1 glutathione peroxidase 1 isoform 1, Heat shock protein 86 (Fragment), HSPA4 Heat shock 70 kDa protein 4, HSPC159 Galectin-related protein, Hypothetical protein, Hypothetical protein DKFZp761K0511, Hypothetical protein FLJ25678, IDH2 Isocitrate dehydrogenase [NADP]-mitochondrial precursor, Integrin-linked protein kinase 1, Isoform 1 of Alpha-parvin, Isoform 1 of Gelsolin precursor, Isoform 1 of Heat shock cognate 71 kDa protein, Isoform 1 of Vinculin, Isoform 2 of Unc-112-related protein 2, Isoform Beta-3B of Integrin beta-3 precursor, Isoform M1 of Pyruvate kinase isozymes M1/M2, Isoform SERCA3B of Sarcoplasmic/endoplasmic reticulum calcium ATPase 3, KCNK15 Potassium channel subfamily K member 15, Lactate dehydrogenase A, Leukocyte elastase inhibitor, L-lactate dehydrogenase B chain, LOC390006 similar to peptidylprolyl isomerase A isoform 1, LTBP1 Latent-transforming growth factor beta-binding protein-isoform 1L precursor, LYAR Cell growth-regulating nucleolar protein, MAPRE2 Isoform 1 of Microtubule-associated protein RP/EB family member 2, MRPS30 28S ribosomal protein S31-mitochondrial precursor, Multimerin-1 precursor, Myosin regulatory light chain, Myosin regulatory light polypeptide 9 isoform b, Myosin-9, NME1 Nucleoside diphosphate kinase A, Peptidyl-prolyl cis-trans isomerase, Peptidylprolyl isomerase B precursor, Phosphoglycerate kinase 1, PINCH protein, Platelet glycoprotein V precursor, Pleckstrin, Protein disulfide-isomerase precursor, Protein DJ-1, PTGS1 Cyclooxygenase 1b3, Rab GDP dissociation inhibitor alpha, RAB6B Ras-related protein Rab-6B, RAC2 Ras-related C3 botulinum toxin substrate 2 precursor, Ras suppressor protein 1, Ras-related protein Rab-11B, Ras-related protein Rab-27B, RcTPI1 (Fragment), Rho GDP-dissociation inhibitor 2, RTN4 Isoform 1 of Reticulon-4, S100A4 Protein S100-A4, SELP P-selectin precursor, SEPT11 Septin-11, Serum deprivation-response protein, SLC2A3 Solute carrier family 2-facilitated glucose transporter member 3, SOD1 16 kDa protein, SPARC SPARC precursor, SPTBN5 Spectrin beta chain-brain 4, STXBP2 Syntaxin-binding protein 2, Superoxide dismutase [Mn]-mitochondrial precursor, TBXAS1 thromboxane A synthase 1 (platelet, cytochrome P450, family 5, subfamily A) isoform TXS-II, Thrombospondin-I precursor, Thrombospondin-2 precursor, TPI1 Isoform 2 of Triosephosphate isomerase, TPM1 tropomyosin 1 alpha chain isoform 7, Transgelin-2, Transitional endoplasmic reticulum ATPase, Tropomyosin 4, Tubulin alpha-1 chain, Tubulin beta-1 chain, Tubulin beta-2C chain, VASP Vasodilator-stimulated phosphoprotein, ANK1 Isoform Er1 of Ankyrin-1, SPTA1 Spectrin alpha chain-erythrocyte, EPB41 Isoform 1 of Protein 4.1, Actin-like protein 2, Isoform 1 of Filamin-C, Filamin A alpha, F-actin capping protein alpha-1 subunit, PDZ and LIM domain protein 1, Fructose-bisphosphate aldolase A, Band 3 anion transport protein, EPB42 Isoform Long of Erythrocyte membrane protein band 4.2, Isoform 1 of F-actin capping protein subunit beta, PKLR Isoform R-type of Pyruvate kinase isozymes R/L, 19 kDa protein, ALB protein (22434), ALB Protein (216773), Apolipoprotein A-I precursor, Apolipoprotein A-IV precursor, Apolipoprotein E precursor, Apolipoprotein F precursor, C4b-binding protein alpha chain precursor, Carbonic anhydrase 1, Clusterin precursor, Complement C4-A precursor, Factor VII active site mutant immunoconjugate, FLJ00385 protein (Fragment), Galectin-3-binding protein precursor, Hypothetical protein DKFZp686I04196 (Fragment), Ig kappa chain V-III region HAH precursor, Ig mu heavy chain disease protein, IGHA1 protein, IGHM protein, IGKV1-5 protein, IGLC1 protein, Isoform 2 of Reelin precursor, PREDICTED: HEG homolog 1, PRO2275, Syntenin-1, Transferrin receptor protein 1, Vitronectin precursor, and von Willebrand factor precursor.
